(19) United States
(12) Patent Application Publication
(10) Pub. No.: US 2007/0280944 A1
(43) Pub. Date: Dec. 6, 2007

(54) Use of 5,10-Methylene Tetrahydrofolate for the Treatment of Cancer

(75) Inventors: Joan M. Robbins, San Diego, CA (US); Mark J. Cantwell, San Diego, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Adventrx Pharmaceuticals, Inc., San Diego, CA (US)

(21) Appl. No.: 10/594,850

(22) PCT Filed: Apr. 1, 2005
(86) PCT No.: PCT/US05/11046

§ 371 Date: Jun. 20, 2007

Related U.S. Application Data
(60) Provisional application No. 60/558,889, filed on Apr. 2, 2004.
(60) Provisional application No. 60/625,479, filed on Nov. 4, 2004.
(60) Provisional application No. 60/658,745, filed on Mar. 4, 2005.

(51) Int. Cl. A61K 39/395; A61K 31/525; A61K 31/7072; A61K 31/704; A61K 31/4745; A61K 31/337

(52) U.S. Cl. 424/145.1; 514/251; 514/49; 514/269; 514/109; 514/410; 514/449; 514/283; 514/34; 514/492

(57) ABSTRACT

The present invention provides novel uses and compositions for 5,10-methylene tetrahydrofolate (“5,10-CH₂-THFA”) in the treatment of cancer. The present invention is based on the surprising result that 5,10-CH₂-THFA, while increasing the efficacy of 5-fluoruracil (5-FU) in reducing the rate of tumor growth and increasing survivorship, also reduces the toxicity to the patient of 5-FU. The present invention provides methods and compositions for treating cancer patients that include 5-FU, 5,10-CH₂-THFA, and one or more additional anticancer drugs. Such methods and compositions can provide increased efficacy and reduced toxicity when compared with current treatment modalities.

This application claims priority to U.S. Provisional application No. 60/558,889 filed Apr. 2, 2004 entitled “Methods of Using 5,10-Methylene Tetrahydrofolate to Treat Cancer”, naming Mark Cantwell and Joan Robbins as inventors; to U.S. Provisional application No. 60/625,479, filed Nov. 4, 2004 entitled “Methods of Using 5,10-Methylene Tetrahydrofolate to Treat Cancer” naming Mark Cantwell and Joan Robbins as inventors; and to U.S. Provisional application No. 60/658,745, filed Mar. 4, 2005 entitled “Methods of using 5,10-methylene hydrofolate in combination therapies to treat cancer” naming Mark Cantwell and Joan Robbins as inventors.

### BACKGROUND OF THE INVENTION

Cancer is a major public health concern. Colorectal cancer alone causes approximately 50,000 deaths per year in the United States. Nearly half of the approximately 130,000 cases of colorectal cancer that are diagnosed every year present with or develop into metastatic disease, for which chemotherapy is the only treatment. New effective drug-based therapies for treatment are urgently sought not only for colorectal cancers, but for other cancers such as, for example, breast cancer, pancreatic cancer, stomach cancers, hepatic cancer, bladder cancer, cervical cancer, head and neck cancers, lung cancers, ovarian cancer, and prostate cancer.

The anticancer drug 5-fluorouracil (5-FU) is converted in the body to FdUMP, an inhibitor of thymidylate synthase (TS), an enzyme required for nucleic acid biosynthesis. 5-FU is commonly used to treat cancers such as colorectal and breast cancer, as well as head and neck cancer, pancreatic cancer, stomach cancer, and non-small-cell lung cancer. 5-FU is commonly used in conjunction with folinic acid (FA, leucovorin), which is converted intracellularly into reduced forms of folate (5,10-methylene tetrahydrofolate or polyglutamates of 5,10-methylene tetrahydrofolate), that are cofactors for thymidylate synthase. The combination of 5-FU and leucovorin has been found to have increased anti-tumor effects when compared with the use of 5-FU alone. 5-FU and 5-FU in combination with leucovorin have been used in combination with other anticancer agents to improve survivorship of patients having recurrent colorectal, breast, stomach, or other cancers.

5,10-methylene tetrahydrofolate (“5,10-CH₂-THFA”) is a reduced folate that can act as a cofactor for thymidylate synthase, either directly or after conversion to its polyglutamates.

Toxicities associated with 5-FU include stomatitis, mucositis, gastrointestinal symptoms, and hematological toxicity, particularly neutropenia, thrombocytopenia, and leucopenia. Toxicity can limit the treatment available to the patient, either by limiting the dosages of anti-cancer agents or by limiting the armory available to the clinician in treating the cancer patient. Thus there is a need to develop improved anti-cancer drug regimens having reduced toxicity that are effective in prolonging survivorship of the patient.

### BRIEF SUMMARY OF THE INVENTION

The present invention provides novel uses for 5,10-methylene tetrahydrofolate (“5,10-CH₂-THFA”) in the treatment of cancer which provide reduced toxicity to the patient and greater efficacy than current modalities.

The present invention is based on the surprising result that 5,10-CH₂-THFA, while increasing the efficacy of 5-FU in reducing the rate of tumor growth and increasing survivorship, also reduces the toxicity to the patient of 5-FU. As disclosed herein, treatment with 5,10-CH₂-THFA and 5-FU reduces tumor growth rate and increases survivorship of tumor-bearing animals with respect to treatment with either 5-FU alone, or 5-FU in combination with leucovorin (folinic acid; FA), while demonstrating less toxicity than either treatment.

The present invention is further based on the finding that treatment of tumor-bearing animals with 5,10-CH₂-THFA and 5-FU and additional anticancer drugs can also improve outcomes with respect to single modality treatments or alternative combination treatments that include the use of 5-FU with leucovorin.

In one aspect the invention provides methods of treating cancer patients with combination chemotherapy that includes 5-fluorouracil (5-FU) or an analog or prodrug of 5-FU; 5,10-CH₂-THFA; and one or more additional anti-cancer drugs. The one or more additional anticancer drugs can be one or more chemotherapeutic agents of any type, including but not limited to chemotherapeutic agents that comprise specific binding members, proteins, nucleic acids, lipids, steroids, large molecules, small molecules, or metals. The one or more anticancer drugs can comprise one or more of: alkylating agents, antimetabolites, mitotic inhibitors, topoisomerase inhibitors, microtubule disrupting drugs, nucleic acid synthesis inhibitors, kinase inhibitors, hormone blocking drugs, proteosome inhibitors, vascularization inhibitors, immune modulators, anti-inflammatory drugs, cytokines, inhibitors of cytokines, receptor-binding drugs, or 5-FU modulators. The method includes: administering 5-FU or an analog or prodrug thereof, 5,10-CH₂-THFA, and at least one additional anticancer drug to a patient with cancer.

In a second aspect of present invention provides compositions for the treatment of cancer that comprise: 5-FU or an analog or prodrug thereof, 5,10-CH₂-THFA, and at least one additional anticancer drug. The one or more additional anticancer drugs can be one or more chemotherapeutic agents of any type, including but not limited to chemotherapeutic agents that comprise specific binding members, proteins, nucleic acids, lipids, steroids, large molecules, small molecules, or metals. The one or more anticancer drugs can comprise one or more of: alkylating agents, antimetabolites, mitotic inhibitors, topoisomerase inhibitors, microtubule disrupting drugs, nucleic acid biosynthesis inhibitors, kinase inhibitors, hormone blocking drugs, proteosome inhibitors, vascularization inhibitors, immune modulators, anti-inflammatory drugs, cytokines, inhibitors of cytokines, receptor-binding drugs, or 5-FU modulators. A multidrug composition of the present invention can be provided in one or more than one formulation.

A third aspect of the present is the use of 5,10-CH₂-THFA in combination with 5-FU or an analog or prodrug thereof and at least one additional chemotherapeutic agent in the manufacture of a composition for the treatment of cancer where the at least one additional chemotherapeutic agent is selected from the group consisting of: alkylating agents, antimetabolites, topoisomerase inhibitors, microtubule disrupting drugs, nucleic acid biosynthesis inhibitors, kinase inhibitors, hormone blocking drugs, proteosome inhibitors, vascularization inhibitors, immune modulators, anti-inflammatory drugs, cytokines, inhibitors of cytokines, receptor-binding drugs, or 5-FU modulators. The use includes manufacturing the pharmaceutical composition as a single formulation or as more than one formulation.

In a fourth aspect, the present invention provides methods for decreasing the toxicity to a patient of a cancer drug treatment regimen that includes administration of 5-FU or an analog or prodrug of 5-FU to a cancer patient by co-administering 5,10-CH₂-THFA.

In some preferred embodiments of this aspect, the present invention includes methods for decreasing toxicity of an analog or prodrug of 5-FU, such as, but not limited to capecitabine, by co-administering 5,10-CH₂-THFA.

In some preferred embodiments of this aspect, the present invention includes methods for decreasing the toxicity of an anticancer treatment that comprises administering 5-FU or an analog or prodrug of 5-FU and an additional anticancer drug (other than 5-FU or a folate cofactor of thymidylate synthase) to a patient with cancer by co-administering 5,10-CH₂-THFA.

A fifth aspect of the present invention is a method of reducing the toxicity to a patient of a anticancer drug treatment regimen that includes 5-FU or an analog or prodrug of 5-FU and leucovorin, comprising substituting 5,10-5,10-CH₂-THFA for leucovorin in the anticancer drug regimen.

In some preferred embodiments of this aspect, the present invention includes methods for decreasing toxicity of an anticancer drug regimen that includes an analog or prodrug of 5-FU, such as, but not limited to, capecitabine, and leucovorin where toxicity of the regimen is decreased by substituting 5,10-CH₂-THFA for leucovorin in the regimen.

In some preferred embodiments of this aspect, the present invention includes methods for decreasing the toxicity of an anticancer treatment that comprises 5-FU or an analog or prodrug of 5-FU, leucovorin, and at least one additional anticancer drug (other than 5-FU or a folate cofactor of thymidylate synthase) to a patient with cancer by substituting 5,10-5,10-CH₂-THFA for leucovorin in the drug regimen.

In a sixth aspect, the present invention provides methods for increasing the efficacy of a cancer drug treatment regimen that includes administration of 5-FU or an analog or prodrug of 5-FU to a cancer patient by co-administering 5,10-5,10-CH₂-THFA.

In some preferred embodiments of this aspect, the present invention includes methods for increasing the efficacy of an analog or prodrug of 5-FU, such as, but not limited to capecitabine, by co-administering 5,10-CH₂-THFA.

In other preferred embodiments of this aspect, the present invention includes methods for increasing the efficacy of an anticancer treatment that comprises administering 5-FU or an analog or prodrug of 5-FU and an additional anticancer drug (other than 5-FU or a folate cofactor of thymidylate synthase) to a patient with cancer by co-administering 5,10-CH₂-THFA.

A seventh aspect of the present invention is a method of increasing the efficacy to a patient of a anticancer drug treatment regimen that includes 5-FU or an analog or prodrug of 5-FU and leucovorin, comprising substituting 5,10-5,10-CH₂-THFA for leucovorin in the anticancer drug regimen.

In some preferred embodiments of this aspect, the present invention includes methods for increasing efficacy of an anticancer drug regimen that includes an analog or prodrug of 5-FU, such as, but not limited to, capecitabine, and leucovorin where efficacy of the regimen is increased by substituting 5,10-CH₂-THFA for leucovorin in the regimen.

In some preferred embodiments of this aspect, the present invention includes methods for increasing the efficacy of an anticancer treatment that comprises 5-FU or an analog or prodrug of 5-FU, leucovorin, and at least one additional anticancer drug (other than 5-FU or a folate cofactor of thymidylate synthase) to a patient with cancer by substituting 5,10-CH₂-THFA for leucovorin in the drug regimen.

In a related aspect, the invention provides a method of increasing the dose of 5-FU or an analog or prodrug of 5-FU in an anticancer drug regimen that includes 5-FU and leucovorin. The method includes: obtaining an anticancer drug regimen that includes 5-FU or an analog or prodrug of 5-FU and leucovorin; substituting 5,10-5,10-CH₂-THFA for leucovorin in the anticancer drug regimen; and increasing the dosage of 5-FU or an analog or prodrug of 5-FU in the anticancer drug regimen. In this aspect, substituting 5,10-CH₂-THFA for leucovorin in the anticancer while increasing the dosage of 5-FU can increase the efficacy of a treatment without prohibitively increasing toxicity.

In yet another related aspect, the invention provides a method of increasing the dose of an additional anticancer drug in an anticancer drug regimen that comprises 5-FU or an analog or prodrug of 5-FU, leucovorin, and an additional anticancer drug. The method includes: obtaining an anticancer drug regimen that includes 5-FU or an analog or prodrug of 5-FU, leucovorin, and at least one additional anticancer drug (other than 5-FU or an analog or prodrug of 5-FU or a folate cofactor of thymidylate synthase); substituting 5,10-CH₂-THFA for leucovorin in the anticancer drug regimen; and increasing the dosage of the one or more additional anticancer drugs in the anticancer drug regimen. In this aspect, substituting 5,10-CH₂-THFA for leucovorin in the anticancer while increasing the dosage an additional anticancer drug used in the regimen can increase the efficacy of a treatment without prohibitively increasing toxicity.

### BRIEF DESCRIPTION OF THE DRAWINGS

FIG. 1 is a graph depicting growth kinetics of HT-29 tumor in Nude mice treated with combinations of 5-FU; 5,10-CH₂-THFA, here represented as “CoFactor”; anti-VEGF (Avastin); and leucovorin. HT-29 tumor volumes were plotted against time from treatment initiation with the indicated drugs. Mean tumor volume±standard error of the mean are plotted. Curves were generated by best-fit analysis.

FIG. 2 is a graph depicting growth kinetics of HT-29 tumor in Nude mice treated with combinations of 5-FU; 5,10-CH₂-THFA, here represented as “CoFactor”; and oxaliplatin. HT-29 tumor volumes were plotted against time from treatment initiation with the indicated drugs. Mean tumor volume±standard error of the mean are plotted. Curves were generated by best-fit analysis.

FIG. 3 is a graph depicting mean tumor volumes following treatment of Nude mice bearing HT-29 tumor with combinations of 5-FU; 5,10-CH₂-THFA, here represented as “CoFactor”; anti-VEGF (Avastin); and leucovorin. Mean tumor volumes 22 days following treatment initiation were plotted for each treatment group. Error bars represent standard error of the means.

FIG. 4 is a graph depicting mean tumor volumes following treatment of Nude mice bearing HT-29 tumor with combinations of 5-FU; 5,10-CH₂-THFA, here represented as “CoFactor”; and oxaliplatin. Mean tumor volumes 22 days following treatment initiation were plotted for each treatment group. Error bars represent standard error of the means.

FIG. 5 depicts Kaplan-Meier plots of survival of Nude mice bearing HT-29 tumor following treatment with combinations of 5-FU; 5,10-CH₂-THFA, here represented as “CoFactor”; leucovorin; and anti-VEGF (Avastin).

FIG. 6 depicts Kaplan-Meier plots of survival of Nude mice bearing HT-29 tumor following treatment with combinations of 5-FU; 5,10-CH₂-THFA, here represented as “CoFactor”; and oxaliplatin.

FIG. 7 is a graph depicting HT-29 tumor growth kinetics in Nude mice treated with combinations 5-FU; 5,10-CH₂-THFA, here represented as “CoFactor”; leucovorin; and anti-VEGF (Avastin). HT-29 tumor volumes were plotted against time from treatment initiation. Mean tumor volume±standard error of the mean are plotted. Curves were generated by best-fit analysis.

FIG. 8 is a graph depicting mean tumor volumes following treatment of Nude mice bearing HT-29 tumor with combinations of 5-FU; 5,10-CH₂-THFA, here represented as “CoFactor”; leucovorin; and anti-VEGF (Avastin). Mean tumor volumes 19 days following treatment initiation were plotted for each treatment group. Error bars represent standard error of the means.

FIG. 9 is a Kaplan-Meier plot of survival of Nude mice bearing HT-29 tumor following treatment with combination of 5-FU; 5,10-CH₂-THFA, here represented as “CoFactor”; and anti-VEGF (Avastin).

FIG. 10 is a Kaplan-Meier plot of survival of Balb/c mice following treatment with 5-FU, 5-FU/leucovorin, and 5-FU/5,10-CH₂-THFA (5,10-CH₂-THFA is labeled as “CoFactor”). FIG. 11 is a graph depicting blood analysis of Balb/c mice following treatment with 5-FU, 5-FU/leucovorin, and 5-FU/5,10-CH₂-THFA (5,10-CH₂-THFA is labeled as “CoFactor”). Blood measurements taken 1 week after drug therapy were divided by the pre-treatment blood measurements to calculate the percentage baseline measurement plotted in the graph. Mean data values±standard errors of the means are plotted for each treatment group. WBC, white blood cells; RBC, red blood cells; HGB, hemoglobin; HCT, hematocrit; MCV, mean cell volume; MCH, mean cell hemoglobin; MCHC, mean cell hemoglobin content; PLT, platelets.

FIG. 12 is a graph depicting platelet toxicity grading of Balb/c mice following treatment with 5-FU, 5-FU/leucovorin, and 5-FU/5,10-CH₂-THFA (5,10-CH₂-THFA is labeled as “CoFactor”). One week following drug treatment, the grade of platelet toxicity was calculated for each mouse. The percentages of mice with grade 1 or 2, grade 3, and grade 4 toxicity are plotted for each treatment group.

FIG. 13 is a graph depicting neutrophil toxicity grading of Balb/c mice following treatment with 5-FU, 5-FU/leucovorin, and 5-FU/5,10-CH₂-THFA (5,10-CH₂-THFA is labeled as “CoFactor”). One week following drug treatment, the grade of neutrophil toxicity was calculated for each mouse. The percentages of mice with grade 1 or 2, grade 3, and grade 4 toxicity in each treatment group are plotted.

FIG. 14 is a graph depicting neutrophil toxicity analysis of Balb/c mice following treatment with 5-FU, 5-FU/leucovorin, and 5-FU/5,10-CH₂-THFA (5,10-CH₂-THFA is labeled as “CoFactor”). One week following drug treatment, mice with grade 4 neutrophil toxicity were subdivided based on their absolute neutrophil counts. The percentages of these mice with the indicated neutrophil cell counts are plotted for each treatment group.

FIG. 15 is a graph depicting weight loss toxicity grading of Balb/c mice following treatment with combinations of 5-FU; leucovorin; 10-CH₂-THFA, here labeled as “CoFactor”; and gemcitabine. One week following drug treatment, the grade of weight loss toxicity was calculated for each mouse. The percentages of mice with grade 0, 1, 2, and 3 toxicity are plotted for each treatment group. Gem=Gemcitabine

FIG. 16 is a graph depicting percent weight loss of Balb/c mice following treatment with combinations of 5-FU; leucovorin; 10-CH₂-THFA, here labeled as “CoFactor”; and gemcitabine. One week following drug treatment, the percentage weight loss from the starting baseline weights were calculated for each mouse. The percentages of mice that fell with the ranges of weight loss indicated in the legend were then plotted for each treatment group. Gem=Gemcitabine

FIG. 17 is a Kaplan-Meier survival plot of Balb/c mice following treatment with combinations of 5-FU; leucovorin; 10-CH₂-THFA, here labeled as “CoFactor”; and gemcitabine. Gem=Gemcitabine

FIG. 18 is a graph depicting lymphopenia toxicity grading of Balb/c mice following treatment with 5-FU, 5-FU/leucovorin, and 5-FU/5,10-CH₂-THFA (5,10-CH₂-THFA is labeled as “CoFactor”). One week following drug treatment, the grade of lymphopenia was calculated for each mouse. The percentages of mice with grade 1/2, grade 3, and grade 4 toxicity are plotted for each treatment group.

FIG. 19 is a graph depicting HT-29 tumor growth kinetics in Nude mice treated with capecitabine (Xeloda), Xeloda/leucovorin, and Xeloda/5,10-CH₂-THFA (5,10-CH₂-THFA is labeled as “CoFactor”). HT-29 tumor volumes were plotted against time from treatment initiation. Mean tumor volume±standard error of the mean are plotted. Curves were generated by best-fit analysis.

FIG. 20 is a Kaplan-Meier survival plot of Balb/c mice following treatment with capecitabine (Xeloda), Xeloda/leucovorin, and Xeloda/5,10-CH₂-THFA (5,10-CH₂-THFA is labeled as “CoFactor”).

FIG. 21 is a graph depicting weight loss toxicity of Balb/c mice following eight days of treatment with capecitabine (Xeloda), Xeloda/leucovorin, and Xeloda/5,10-CH₂-THFA (5,10-CH₂-THFA is labeled as “CoFactor”).

### DETAILED DESCRIPTION OF THE INVENTION

### Definitions

An “anticancer drug” is any drug used in the treatment of cancer. Some nonlimiting examples of some investigational anticancer drugs that can be used in the methods and compositions of the present invention are provided in Table 1.

A “chemotherapeutic agent” is any chemical entity having biological activity and useful in the treatment of disease. In cancer therapy, a chemotherapeutic agent is a chemical entity that directly or indirectly causes the death of cancer cells. A chemotherapeutic agent can have anti-cancer effects either as a single agent or in combination with one or more other chemotherapeutic agents.

An “analog” of an anticancer drug or chemotherapeutic agent is a chemical compound that is structurally similar to the anticancer drug or chemotherapeutic agent but differs slightly in composition (as in the replacement of one atom by an atom of a different element or the addition or substitution of a particular functional group). As used herein, “analog” can also mean a chemical compound the is structurally similar or identical but also includes additional moieties that can, for example, enhance solubility, retard degradation, increase half-life in the circulation, confer membrane permeability, or direct tissue or cellular targeting, for example. Preferably, an analog of a compound, anticancer drug or chemotherapeutic agent has essentially the same activity as the compound, anticancer drug or chemotherapeutic agent when administered to the patient in a therapeutically effective amount.

A “prodrug” of an anticancer drug or chemotherapeutic agent is a molecule which is converted within the body to the anticancer drug or therapeutic agent but on its own either has no activity or has an activity quantitatively or qualitatively different from that of the anticancer drug.

As used herein, an “anticancer drug regimen”, a “chemotherapy drug regimen”, an “anticancer drug protocol,” or a “chemotherapy protocol” is a formal outline or plan of what treatments a cancer patient will receive and exactly when and in what dosages each should be given.

As used herein, a “folate cofactor of thymidylate synthase” or a “folate cofactor of TS” is a reduced folate molecule such as 5,10-CH₂-THFA or a polyglutamate of 5,10-CH₂-THFA that can enhance the inhibition of thymidylate synthase by 5-FU. As used herein a “folate cofactor of thymidylate synthase” can also be a precursor or prodrug of a folate molecule that enhances the inhibition of thymidylate synthase. For example, a folate cofactor such as folinic acid (5-formyl-tetrahydrofolate, leucovorin) can be converted to 5,10-CH₂-THFA and polyglutamates of 5,10-CH₂-THFA in the body.

“Toxicity” refers to harmful effects of an entity on the cells, tissues, organs, or systems of the body. Toxic effects result from biochemical reactions of the entity with the cells or tissues of the subject being treated, and can be general or specific, involving a particular system or organ. Toxicity can include, as nonlimiting examples, increased lacrimation; mucositis; esophagopharyngitis; neurological toxicity, such as parasthesias, insomnia, and dizziness; gastrointestinal toxicity, such as nausea, vomiting, and diarrhea; weight loss toxicity; cardiac toxicity; dermatological toxicity, including alopecia, sweating, and rashes; and hematological toxicity, such as, but not limited to, neutropenia, thrombocytopenia, lymphopenia, and leucopenia. Clinical definitions of toxicity parameters can be found in the National Cancer Institute's Common Toxicity Criteria (version 3) or in the World Health Organization Toxicity Criteria.

“Efficacy” of an anticancer treatment or chemotherapy regimen is determined by its anti-tumor or anti-cancer cell effects and ability to improve clinical results of treatment, such as, for example, remission, time to progression, response rate, and survivorship. Accepted methods of assessing the efficacy of an anticancer treatment or chemotherapy regimen are well-established in the field of cancer treatment. For example, anti-cancer effects can be assessed by detecting cancer cells or markers, for example in serum or plasma. Examples of tumor proteins or antigens that can be detected include CEA for colon cancer and CA 19-9 for pancreatic cancer. For solid tumors, anti-tumor effects can be measured by monitoring tumor size and the change in tumor size over time. In clinical studies, number of lesions, tumor size, and tumor growth rate can be monitored by radiography, tomography, and, where possible, direct measurement of tumor mass. Antitumor effects can also be measured using molecular biology and biochemistry techniques, such as ELISA, PCR, western blotting, or immunocytochemistry.

TABLE 1
Investigational Colorectal Drugs

| Cate- | | | |
|---|---|---|---|
| gory | Drug | Company | Mechanism |
| 1 | ABT-751 | Abbott | Microtubulin inhibitor |
| | | Laboratories | |
| 1 | Epothilone D | Kosan | Microtubulin Inhibitor |
| | | Biosciences | |
| 2 | 105AD7 | Onyvax | Anti-idiotype vaccine |
| 2 | BCG | Intracel | Mycobacterium |
| | | | Autologous Vaccine |
| 2 | EP2101 | Epimmune | Peptide Vaccine |
| 2 | Mutant ras + | NCI | Dendritic vaccine |
| | IL-2 vaccine | | |
| 2 | SGN-00101 | Stressgen | BCG vaccine |
| 3 | ABX-EGF | Abgenix | Anti-EGFR |
| | (panitumumab) | | |
| 3 | GW572016 | GlaxoSmithKline | EGFR/ERBb2 |
| | | | inhibitor |
| 3 | BAY 43-9006 | Bayer/Onyx | RAF/VEGF signal |
| | | | inhibitor |
| 4 | EKB-569 | Wyeth-Ayerst | EGF Receptor |
| | | | kinase inhibitor |
| 4 | Erlotinib | Genentech | Tyrosine kinase |
| | | | inhibitor |
| 4 | Gefitinab (Iressa) | AstraZeneca | EGFR tyrosine |
| | | | kinase inhibitor |
| 4 | PTK787/ZK 222584 | Novartis | VEGFR Tyrosine |
| | | | Kinase Inhibitor |
| 4 | E7070 | Eisai Medical | Cdk2 and cyclin E |
| | | Research | inhibitor |
| 5 | Celecoxib (Celebrex) | Pfizer | Nonsteroidal Anti- |
| | | | inflammatory |
| 5 | Rofecoxib (Vioxx) | Merck | Nonsteroidal Anti- |
| | | | inflammatory |
| 6 | GM-CSF | | Cytokine |
| 6 | Interferon alpha | | Cytokine |
| 6 | Interferon beta | | Cytokine |
| 6 | TNFerade | Genvec | Adenovirus TNF |
| | | | Cytokine |
| 7 | DAVANAT | Pro- | Carbohydrate binder |
| | | Pharmaceuticals | that targets 5-FU |
| | | | to cell |
| 7 | Etoposide | Schering | Farnesyl transferase |
| | | Plough | inhibitor |
| 7 | LMB-9 | NCI | Lewis Y antibody |
| 8 | Imatinib (Gleevec) | Novartis | |
| 8 | Oblimersin | Genta | BCL-2 inhibitor |
| 9 | Tezacitabine | Chiron | Nucleoside |
| | | | Analogue |
| 10 | Antineoplaston | Burzynski | |
| | | Research Inst. | |
| 10 | Mistletoe extract | NCCAM | |
| | (Helixor A) | | |
| 10 | N-phosphonacetyl- | | 5-FU modulator |
| | L-aspartic acid | | |
| | (PALA) | | |
| 10 | PHY906 | PhytoCeutica | Anti-diarrhea |
| 10 | Talaporfin sodium | Light Sciences | Light activated |
| | (LS11) | Corp. | drug |
| 10 | Thalidomide | NCI | Anti-vascular |

1 Microtubulin Inhibitor
2 Vaccine
3 EGFR/VEGFR Target
4 Tyrosine Kinase/Transcription Factor Inhibitor
5 Nonsteroidal Anti-Inflammatory
6 Cytokine
7 Carbohydrate/Lipid
8 Apoptosis Regulator
9 Nucleoside Analogue
10 Miscellaneous

I. Methods of Treating a Patient with Cancer Using a Combination Therapy that Includes 5-FU, 5,10-CH₂-THFA, and at Least One Additional Anticancer Drug

The invention provides methods of treating cancer patients with combination chemotherapy that includes: 5-5-FU or an analog or prodrug of 5-FU; 5,10-CH₂-THFA; and one or more additional anti-cancer drugs. The one or more additional anticancer drugs can be one or more chemotherapeutic agents of any type, including but not limited to chemotherapeutic agents that comprise specific binding members, proteins, nucleic acids or nucleic acid analogs (such as, but not limited to antisense molecules, ribozymes, and siRNAs), lipids, steroids, large molecules, small molecules, or metals. The one or more anticancer drugs can comprise one or more chemotherapeutic agents, such as but not limited to: topoisomerase inhibitors (e.g., irinotecan, topotecan), antimetabolite drugs (e.g., methotrexate, gemcitabine, tezacitabine ), 5-FU modulators, alkylating agents (e.g., cyclophosphamide, carmustine), nucleic acid biosynthesis inhibitors (e.g., mitomycin, doxorubicin, cisplatin, oxaliplatin), microtubule disrupting drugs (e.g., paclitaxel, docetaxel, vinolrebine, vincristine), hormone blocking drugs (e.g., tamoxifen), inhibitors of kinases, including but not limited to receptor and nonreceptor tyrosine kinases (e.g., Iressa, Tarceva, SU5416, PTK787, Gleevec), proteosome inhibitors (e.g., bortezomib), immune modulators (e.g., levamisole), anti-inflammatory drugs, vascularization inhibitors, cytokines (e.g., interleukins, tumor necrosis factors), and drugs that inhibit the activity of cytokines, hormones, or receptors for cytokines or hormones (e.g., the anti-VEGF antibody bevacizumab or “Avastin”). An anticancer drug can also be a drug under investigation for potential anti-cancer activity, such as those listed in Table 1. Anti-cancer drugs include monoclonal antibodies, such as but not limited to monoclonal antibodies that bind cytokines, hormones, or hormone receptors (e.g., antibodies that block activation of EGF or VEGF growth factors, such as Avastin, erbutux, herceptin), etc.

The method includes: administering 5-FU or an analog or prodrug thereof, 5,10-CH₂-THFA; and at least one additional anticancer drug to a patient with cancer. As used herein, an “additional” anti-cancer drug is an anti-cancer drug that is not 5,10-CH₂-THFA, 5-FU or an analog or prodrug of 5-FU, or leucovorin.

A cancer patient can be a patient with any type of cancer. In some preferred embodiments of the present invention, the patient has a tumor type that in current practice is commonly treated with 5-FU, such as, for example, colorectal carcinoma, pancreatic, breast, head and neck, esophageal cancer, or stomach cancer. In some preferred embodiments of the present invention, the patient has a tumor type that in current practice is not commonly treated with 5-FU, such as, but not limited to ovarian cancer or cervical cancer. The inventors also contemplate that combination therapies that use 5,10-CH₂-THFA, 5-FU (or an analog or prodrug thereof), and one or more additional anti-cancer drugs have potential for treating cancers other than those currently commonly treated with 5-FU.

Those skilled in the art of cancer treatment and chemotherapy would be able to determine optimal dosages and regimens for 5,10-CH₂-THFA and 5-FU using well-established protocols for evaluating toxicity and efficacy. Some preferred treatments of cancer patients with 5-FU and 5,10-CH₂-THFA are regimens using from 10 milligrams to 1 gram of 5,10-CH₂-THFA per m², preferably from 20 milligrams to 500 milligrams of 5,10-CH₂-THFA per m², and more preferably from about 30 milligrams to about 250 milligrams of 5,10-CH₂-THFA per m². For example, a preferred dose of 5,10-CH₂-THFA can be from about 30 to about 120 milligrams per m². The foregoing are general guidelines only that can be expanded or altered based on for example, cancer type and grade, patient age, health status, and sex, the particular drugs used in combination, the route and frequency of administration, and experimental and clinical findings using a multidrug combination.

Dosage of 5-FU can be from about 25 milligrams to about 5 grams per m², and is preferably from about 50 milligrams to 2.5 grams per m², and more preferably from about 100 milligrams to about 1 gram per m². For example, a preferred dose of 5-FU can be from about 250 to about 700 milligrams per m². The foregoing are general guidelines only that can be expanded or altered based on for example, cancer type and grade, patient age, health status, and sex, the particular drugs used in combination, the route and frequency of administration, and experimental and clinical findings using a multidrug combination. 5-FU can be administered by any feasible means, including injection or IV feed.

In some preferred embodiments, a prodrug or analog of 5-FU is used in combination therapy rather than 5-FU itself. In the tissues of a patient, 5-FU is converted to 5-fluoro-2′-deoxyuridylate (FdUMP) the inhibitor of thymidylate synthase. In the present application, “analog or prodrug of 5-FU” is used to mean an analog or prodrug that can be directly or indirectly converted to an inhibitor of thymidylate synthase, such as FdUMP. One prodrug of 5-FU that can be used in the methods of the present invention is N4-pentoxylcarbonyl-5′-deoxy-5-fluorocytidine (capecitabine). In one preferred embodiment, the method of the present invention comprises administering N4-pentoxylcarbonyl-5′-deoxy-5-fluorocytidine (capecitabine); 5,10-CH₂-THFA; and at least one additional anticancer drug to a patient with cancer. The dosage of capecitabine can be determined by skilled clinicians and depends in part on the frequency of administration. For example, the of daily dosage of capecitabine can be from about 500 mg to about 7500 mg per m², preferably from about 1000 mg to about 5000 mgs per m², and more preferably from about 1500 mg to about 3000 mg per m². The dose can be divided into one to six (preferably two) administrations per day. The foregoing are general guidelines only that can be expanded or altered based on for example, cancer type and grade, patient age, health status, and sex, the particular drugs used in combination, the route and frequency of administration, and experimental and clinical findings using a multidrug combination. Capecitabine can be administered by any feasible means including injection, IV feed, or in an oral formulation.

An analog combination that can be used in the methods of the present invention is Tegafur (TF) and uracil (U) used in a 1:4 combination known as UFT. In one preferred embodiment, the method of the present invention comprises administering UFT; 5,10-CH₂-THFA; and at least one additional anticancer drug to a patient with cancer. The dosage of UFT can be determined by skilled clinicians and depends in part on the frequency of administration. For example, the daily dosage of UFT can be from about 50 mg to about 3000 mg per m², preferably from about 100 mg to about 2000 mg per m², and more preferably from about 200 mg to about 1000 mg per m². Anticancer regimens that include UFT can optionally also include calcium folinate administered with UFT. The foregoing are general guidelines only that can be expanded or altered based on for example, cancer type and grade, patient age, health status, and sex, the particular drugs used in combination, the route and frequency of administration, and experimental and clinical findings using a multidrug combination. UFT can be administered by any feasible means, including injection, IV feed, or in an oral formulation.

Dosage for the one or more additional anticancer drugs used in a multidrug regimen of the present invention can also be determined by studies using escalating dosages and monitoring of toxicity and efficacy. In determining dosages of an anticancer drug to be used in combination therapy that have been used independently in chemotherapy regimens, practitioners can take into account dosages of drugs used in established chemotherapy regimens.

The reduced toxicity of 5-FU (or an analog or prodrug thereof) when combined with 5,10-CH₂-THFA can permit drug regimens in which 5,10-CH₂-THFA and 5-FU (or an analog or prodrug thereof) are used in combination with one or more additional anti-cancer drugs that would be prohibitively toxic in the absence of CH₂-THFA.

The drugs can be administered intravenously, orally, or by any other feasible means, according to regimens that can be determined by qualified clinicians. The anticancer drugs used in the combination protocols of the present invention can be administered separately or one or more of the anticancer drugs used in the combination protocols can be administered together. Where one or more anticancer drug is administered separately, the timing and schedule of administration of each drug can vary.

For example, bolus injection of each drug can be given once weekly for a number of weeks. Preferably, 5,10-CH₂-THFA is administered prior to 5-FU or 5-FU analog or prodrug. For example, the patient can receive the 5,10-CH₂-THFA dose from about 10 minutes to about four hours prior to receiving the 5-FU dose. An additional anticancer drug used in combination therapy can be administered before, during, or after administration of 5-FU (or an analog or prodrug thereof), or can be administered during periods in which the patient does not receive 5-FU (or an analog or prodrug thereof) and 5,10-CH₂-THFA. The protocol for the combination therapy is not limiting, and can include many any feasible administration protocols with respect to frequency, duration, and dosage.

In some embodiments of this aspect of the present invention, treating a cancer patient with 5,10-CH₂-THFA, 5-FU (or an analog or prodrug thereof), and one or more additional anti-cancer drugs can reduce the rate of tumor growth in a cancer patient when compared with treating the patient with the one or more additional anti-cancer drugs in the absence of 5,10-CH₂-THFA and 5-FU (or an analog or prodrug thereof), or when compared with treating a patient with 5-FU (or an analog or prodrug thereof) and the one or more additional anti-cancer drugs in the absence of 5,10-CH₂-THFA.

In some embodiments of this aspect of the present invention, treating cancer patients with 5,10-CH₂-THFA, 5-FU (or an analog or prodrug thereof), and one or more additional anti-cancer drugs can increase the survivorship of cancer patients when compared with treating cancer patients with the one or more additional anti-cancer drugs in the absence of 5,10-CH₂-THFA and 5-FU (or an analog or prodrug thereof) or when compared with treating cancer patients with 5-FU (or an analog or prodrug thereof) and the one or more additional anti-cancer drugs in the absence of 5,10-CH₂-THFA.

II. Compositions for the Treatment of Cancer Comprising 5-FU, 5,10-Methylene Tetrahydrofolate, and at Least One Additional Anticancer Drug

A second aspect of the present invention is compositions for the treatment of cancer that comprise: 5-FU or an analog or prodrug thereof, 5,10-CH₂-THFA, and at least one additional anticancer drug. The one or more additional anticancer drugs can be one or more chemotherapeutic agents of any type, including but not limited to chemotherapeutic agents that comprise specific binding members, proteins, nucleic acids or nucleic acid analogs (such as, but not limited to antisense molecules, ribozymes, and siRNAs), lipids, steroids, large molecules, small molecules, or metals. The one or more anticancer drugs can comprise one chemotherapeutic agents, such as but not limited to: topoisomerase inhibitors (e.g., irinotecan, topotecan), antimetabolite drugs (e.g., methotrexate, gemcitabine), mitotic inhibitors, 5-fluorouracil modulators, alkylating agents (e.g., cyclophosphamide, carmustine), nucleic acid biosynthesis inhibitors (e.g., mitomycin, doxorubicin, cisplatin, oxaliplatin), microtubule disrupting drugs (e.g., paclitaxel, docetaxel, vinolrebine, vincristine), hormone blocking drugs (e.g., tamoxifen), inhibitors of kinases, including but not limited to receptor and nonreceptor tyrosine kinases (e.g., Iressa, Tarceva, SU5416, PTK787, Gleevec), proteosome inhibitors (e.g., bortezomib), immune modulators (e.g., levamisole), anti-inflammatory drugs, vascularization inhibitors, cytokines (e.g., interleukins, tumor necrosis factors), and drugs that inhibit the activity of cytokines, hormones, or receptors for cytokines or hormones (e.g., the anti-VEGF antibody bevacizumab or “Avastin”). An anticancer drug can also be a drug under investigation for potential anti-cancer activity, such as those listed in Table 1. Anti-cancer drugs include monoclonal antibodies, such as but not limited to monoclonal antibodies that bind cytokines, hormones, or hormone receptors (e.g., antibodies that block activation of EGF or VEGF growth factors, such as Avastin, erbutux, herceptin), etc.

The present invention includes anticancer drug combinations that include 5-FU (or an analog or prodrug thereof), 5,10-CH₂-THFA, and one or more additional anticancer drugs formulated as pharmaceutical compositions. An anticancer drug combination can comprise one or more pharmaceutical formulations. For example, 5-FU (or an analog or prodrug thereof), 5,10-CH₂-THFA, and one or more additional anticancer drugs can each be provided as a separate formulation. Alternatively, two or more of 5-FU (or an analog or prodrug thereof), 5,10-CH₂-THFA, and one or more additional anticancer drugs can be provided together in a formulation. Separate formulations that are used in a multidrug anticancer regimen of the present invention can be designed for the same or different routes of administration.

III. Use of 5-FU, 5,10-methylene tetrahydrofolate, and at Least One Additional Anticancer Drug in the Manufacture of a Pharmaceutical Composition for the Treatment of Cancer

The present invention also includes the use of 5-FU or an analog or prodrug thereof, 5,10-CH₂-THFA, and at least one additional anticancer drugs in the manufacture of a pharmaceutical composition for the treatment of cancer. The at least one additional anticancer drug can be any of the following: a topoisomerase inhibitor (e.g., irinotecan, topotecan), an antimetabolite drug (e.g., methotrexate, gemcitabine), a mitotic inhibitor, a 5-Fu modulator, an alkylating agent (e.g., cyclophosphamide, carmustine), a nucleic acid biosynthesis inhibitor (e.g., mitomycin, doxorubicin, cisplatin, oxaliplatin), a microtubule disrupting drug (e.g., paclitaxel, docetaxel, vinolrebine, vincristine), a hormone blocking drug (e.g., tamoxifen), an inhibitor of kinases, including but not limited to receptor and nonreceptor tyrosine kinases (e.g., Iressa, Tarceva, SU5416, PTK787, Gleevec), a proteosome inhibitor (e.g., bortezomib), an immune modulator (e.g., levamisole), an anti-inflammatory drug, a vascularization inhibitor, a cytokine (e.g., interleukins, tumor necrosis factors), and a drug that inhibits the activity of cytokines, a hormone, or a receptor for cytokines or hormones (e.g., bevacizumab or “Avastin”). The use includes manufacturing the pharmaceutical composition as a single formulation or as more than one formulation. For example, 5-FU may be provided as an injectable aliquot and 5,10-CH₂-THFA and at least one additional anticancer drug may be provided as an additional injectable aliquot to be administered prior to the 5-FU dose. Alternatively, 5-FU, 5,10-CH₂-THFA, and at least one additional anticancer drug can all be provided in separate formulations, so that each can be administered separately, and where each drug aliquot is manufactured to have the appropriate dose for a particular combination drug regimen.

The pharmaceutical compositions comprise a pharmaceutically acceptable carrier prepared for storage and preferably subsequent administration, which have a pharmaceutically effective amount of the compound in a pharmaceutically acceptable carrier or diluent. Acceptable carriers or diluents for therapeutic use are well known in the pharmaceutical art, and are described, for example, in Remington's Pharmaceutical Sciences, 18th Ed., Mack Publishing Co., Easton, Pa. (1990)). Preservatives, stabilizers, dyes and even flavoring agents can be provided in the pharmaceutical composition. For example, sodium benzoate, ascorbic acid and esters of p-hydroxybenzoic acid can be added as preservatives. In addition, antioxidants and suspending agents can be used.

Depending on the target tissue, the pharmaceutical compositions of the present invention can be formulated and used as tablets, capsules or solutions for oral administration; salves or ointments for topical application; suppositories for rectal administration; sterile solutions, suspensions, and the like for use as inhalants or nasal sprays. Injectables can also be prepared in conventional forms either as liquid solutions or suspensions, solid forms suitable for solution or suspension in liquid prior to injection, or as emulsions. Suitable excipients are, for example, water, saline, dextrose, mannitol, lactose, lecithin, albumin, sodium glutamate, cysteine hydrochloride and the like. In addition, if desired, the injectable pharmaceutical compositions can contain minor amounts of nontoxic auxiliary substances, such as wetting agents, pH buffering agents and the like.

The pharmaceutically effective amount of a composition required as a dose will depend on the route of administration, the type of cancer being treated, and the physical characteristics of the patient. The dose can be tailored to achieve a desired effect, but will depend on such factors as weight, diet, concurrent medication and other factors which those skilled in the medical arts will recognize. In practicing the methods of the present invention, the pharmaceutical compositions can be used alone, or in combination with other therapeutic or diagnostic agents. The pharmaceutical compositions can be administered to the patient in a variety of ways, including topically, parenterally, intravenously, subcutaneously, intramuscularly, colonically, rectally, nasally or intraperiotoneally, employing a variety of dosage forms. Preferably, the pharmaceutical compositions are administered parenterally, intravenously, or orally. Such methods can also be used in testing the activity of test compounds in vivo.

When administered orally as a suspension, compositions of the present invention are prepared according to techniques well-known in the art of pharmaceutical formulation and may contain microcrystalline cellulose for imparting bulk, alginic acid or sodium alginate as a suspending agent, methylcellulose as a viscosity enhancer, and sweeteners/flavoring agents known in the art. As immediate release tablets, these compositions may contain microcrystalline cellulose, dicalcium phosphate, starch, magnesium stearate and lactose and/or other excipients, binders, extenders, disintegrants, diluents and lubricants known in the art. Components in the formulation of a mouthwash or rinse include antimicrobials, surfactants, cosurfactants, oils, water and other additives such as sweeteners/flavoring agents known in the art.

When administered by a drinking solution, the composition comprises one or more of the compounds of the present invention, dissolved in water, with appropriate pH adjustment, and with carrier. The compound may be dissolved in distilled water, tap water, spring water, and the like. The pH can preferably be adjusted to between about 3.5 and about 8.5. Sweeteners may be added, e.g., 1% (w/v) sucrose.

The formulations of this invention may be varied to include; (1) other acids and bases to adjust the pH; (2) other tonicity imparting agents such as sorbitol, glycerin and dextrose; (3) other antimicrobial preservatives such as other parahydroxy benzoic acid esters, sorbate, benzoate, propionate, chlorbutanol, phenylethyl alcohol, benzalkonium chloride, and mercurials; (4) other viscosity imparting agents such as sodium carboxymethylcellulose, microcrystalline cellulose, polyvinylpyrrolidone, polyvinyl alcohol and other gums; (5) suitable absorption enhancers; (6) stabilizing agents such as antioxidants, like bisulfite and ascorbate, metal chelating agents such as sodium edetate and drug solubility enhancers such as polyethylene glycols.

IV. Methods for Decreasing the Toxicity to a Patient of an Anticancer Drug Treatment Regime that Includes 5-FU

The present invention also provides methods for decreasing the toxicity to a patient of a cancer drug treatment regimen that includes 5-FU, or an analog or prodrug of 5-FU, to a cancer patient by adding 5,10-CH₂-THFA to the drug regimen.

In one aspect, the method comprises: obtaining an anticancer drug protocol that comprises 5-FU or an analogue or prodrug thereof, and adding 5,10-CH₂-THFA to the anticancer drug protocol to obtain an anticancer drug protocol having reduced toxicity to the patient. The method for decreasing the toxicity of a cancer drug treatment that includes administration of 5-FU or an analogue or prodrug thereof comprises administering 5,10-5,10-CH₂-THFA to the patient before, after, or concurrent with the administration of 5-FU to reduce the toxicity of 5-FU. Preferably, administration of 5,10-CH₂-THFA is before administration of 5-FU. In some preferred embodiments of this aspect of the present invention, 5,10-CH₂-THFA is administered to a patient receiving 5-FU to reduce hematological toxicity of 5-FU. In preferred embodiments of this aspect of the present invention, 5-FU and 5,10-CH₂-THFA are administered to the patient in the absence of leucovorin (folinic acid, FA).

A cancer patient can be a patient with any type of cancer. In some preferred embodiments of the present invention in which 5,10-CH₂-THFA is administered to a cancer patient receiving 5-FU, the patient has a tumor type that is currently treated with 5-FU, such as, for example, colorectal carcinoma, pancreatic cancer, breast cancer, head-and-neck cancer, esophageal, or stomach cancer.

The invention is based on the surprising result that 5,10-CH₂-THFA, while increasing the efficacy of 5-FU in reducing the rate of tumor growth and increasing survivorship, also reduces the toxicity of 5-FU towards nontumor cells. As disclosed in Examples 1 and 2, treatment with 5,10-CH₂-THFA and 5-FU reduces tumor growth rate and increases survivorship of tumor-bearing animals with respect to treatment with either 5-FU alone, or 5-FU in combination with leucovorin (folinic acid), while demonstrating less toxicity to the animal than either treatment.

As used herein, “reduce the toxicity” refers to reducing toxic systemic effects on the patient, or toxic effects on the noncancerous cells of the patient. Toxicity can include, as nonlimiting examples, increased lacrimation; mucositis; esophagopharyngitis; neurological toxicity, such as parasthesias, insomnia, and dizziness; gastrointestinal toxicity, such as nausea, vomiting, and diarrhea; weight loss toxicity; cardiac toxicity; dermatological toxicity, including alopecia, sweating, and rashes; and hematological toxicity, such as, but not limited to, neutropenia, thrombocytopenia, lymphopenia, and leucopenia.

In some preferred embodiments of this aspect of the present invention, 5,10-CH₂-THFA is administered in combination therapy with 5-FU to reduce the degree of hematological toxicity associated with 5-FU treatment. For example, administering 5,10-CH₂-THFA along with 5-FU can reduce neutropenia, thrombocytopenia, lymphopenia, or leucopenia associated with chemotherapy regimens that include 5-FU, including but not limited to chemotherapy regimens that include 5-FU and leucovorin (folinic acid).

Those skilled in the art of cancer treatment and chemotherapy would be able to determine optimal dosages and regimens for 5,10-CH₂-THFA and 5-FU using well-established protocols for evaluating toxicity and efficacy. Some preferred treatments of cancer patients with 5-FU and 5,10-CH₂-THFA are regimens using from 10 milligrams to 1 gram of 5,10-CH₂-THFA per m², preferably from 20 milligrams to 500 milligrams of 5,10-CH₂-THFA per m², and more preferably from about 30 milligrams to about 250 milligrams of 5,10-CH₂-THFA per m². For example, a preferred dose of 5,10-CH₂-THFA can be from about 30 to about 120 milligrams per m². The foregoing are general guidelines only that can be expanded or altered based on for example, cancer type and grade, patient age, health status, and sex, the particular drugs used in combination, the route and frequency of administration, and experimental and clinical findings using a multidrug combination.

Dosage of 5-FU can be from about to about 25 milligrams to about 5 grams per m², and is preferably from about 50 milligrams to 2.5 grams per m², and more preferably from about 100 milligrams to about 1 gram per m². For example, a preferred dose of 5-FU can be from about 250 to about 700 milligrams per m². The foregoing are general guidelines only that can be expanded or altered based on for example, cancer type and grade, patient age, health status, and sex, the particular drugs used in combination, the route and frequency of administration, and experimental and clinical findings using a multidrug combination. 5-FU can be administered by any feasible means, including injection or IV feed.

In some preferred embodiments, a prodrug or analog of 5-FU is used in combination therapy rather than 5-FU itself. In the tissues of a patient, 5-FU is converted to 5-fluoro-2′-deoxyuridylate (FdUMP) the inhibitor of thymidylate synthase. In the present application, “analog or prodrug of 5-FU” is used to mean an analog or prodrug that can be directly or indirectly converted to an inhibitor of thymidylate synthase, such as FdUMP. One prodrug of 5-FU that can be used in the methods of the present invention is N4-pentoxylcarbonyl-5′-deoxy-5-fluorocytidine (capecitabine). In one preferred embodiment, the method of the present invention comprises administering N4-pentoxylcarbonyl-5′-deoxy-5-fluorocytidine (capecitabine); 5,10-CH₂-THFA; and at least one additional anticancer drug to a patient with cancer. The dosage of capecitabine can be determined by skilled clinicians and depends in part on the frequency of administration. For example, the of daily dosage of capecitabine can be from about 500 mg to about 7500 mg per m², preferably from about 1000 mg to about 5000 mg per m², and more preferably from about 1500 mg to about 3000 mg per m². The dose can be divided into one to six (preferably two) administrations per day. The foregoing are general guidelines only that can be expanded or altered based on for example, cancer type and grade, patient age, health status, and sex, the particular drugs used in combination, the route and frequency of administration, and experimental and clinical findings using a multidrug combination. Capecitabine can be administered by any feasible means including injection, IV feed, or in an oral formulation.

An analog combination that can be used in the methods of the present invention is Tegafur (TF) and uracil (U) used in a 1:4 combination known as UFT. In one preferred embodiment, the method of the present invention comprises administering UFT; 5,10-CH₂-THFA; and at least one additional anticancer drug to a patient with cancer. The dosage of UFT can be determined by skilled clinicians and depends in part on the frequency of administration. For example, the daily dosage of UFT can be from about 50 mg to about 3000 mg per m², preferably from about 100 mg to about 2000 mg per m², and more preferably from about 200 mg to about 1000 mg per m². Anticancer regimens that include UFT can optionally also include calcium folinate administered with UFT. The foregoing are general guidelines only that can be expanded or altered based on for example, cancer type and grade, patient age, health status, and sex, the particular drugs used in combination, the route and frequency of administration, and experimental and clinical findings using a multidrug combination. UFT can be administered by any feasible means, including injection, IV feed, or in an oral formulation.

Some examples of anticancer drug protocols that use capecitabine are described in Blum JL, et al. “Multicenter phase II study of capecitabine in paclitaxel-refractory metastatic breast cancer.” J Clin Oncol 1999; 17:485-93; in Hoff et al. “Comparison of oral capecitabine versus intravenous fluorouracil plus leucovorin as first-line treatment in 605 patients with metastatic colorectal cancer: results of a randomized phase III study.” J Clin Oncol 2001;19(8):2282-92; and in Van Cutsem E, et al. “Oral capecitabine compared with intravenous fluorouracil plus leucovorin in patients with metastatic colorectal cancer: results of a large phase III study.” J Clin Oncol 2001; 19(21):4097-106; all of which are herein incorporated by reference, in particular for disclosure of chemotherapy regimens using capecitabine. The present invention includes administering 5,10-CH₂-THFA in protocols that include capecitabine to reduce toxicity of capecitabine treatment.

For example, one protocol includes administering capecitabine (1000-1250 mg per m²) twice daily for two weeks, followed by a one week rest period, and then followed by further three week cycles. 5,10-CH₂-THFA can be added to protocols such as these, for example, and the protocols can be optimized based on clinical trials for toxicity and efficacy.

In some preferred embodiments of this aspect, the present invention includes methods for decreasing the toxicity of an anticancer treatment that comprises administering 5-FU or an analog or prodrug of 5-FU and an additional anticancer drug (other than 5-FU or a folate cofactor of thymidylate synthase) to a patient with cancer by co-administering 5,10-5,10-CH₂-THFA. The method includes: obtaining an anticancer drug protocol that comprises 5-fluorouracil or an analogue or prodrug thereof and at least one additional anticancer drug, and adding 5,10-CH₂-THFA to the anticancer drug protocol to obtain an anticancer drug protocol having reduced toxicity to the patient.

An additional anticancer drug can be any type of anticancer drug, including without limitation, a topoisomerase inhibitor (e.g., irinotecan, topotecan), an antimetabolite drug (e.g., methotrexate, gemcitabine), a 5-fluorouracil modulator, an alkylating agent (e.g., cyclophosphamide, carmustine), a nucleic acid biosynthesis inhibitor (e.g., mitomycin, doxorubicin, cisplatin, oxaliplatin, carboplatin), a microtubule disrupting drug (e.g., paclitaxel, docetaxel, vinolrebine, vincristine), a hormone blocking drug (e.g., tamoxifen), a kinase inhibitor, including but not limited to an inhibitor of receptor or nonreceptor tyrosine kinases (e.g., Iressa, Tarceva, SU5416, PTK787, Gleevec), a proteosome inhibitor (e.g., bortezomib), an immune modulator (e.g., levamisole), an anti-inflammatory drug, a vascularization inhibitor, a cytokine (e.g., interleukins, tumor necrosis factors), or a drug that inhibits the activity of a cytokine, hormone, or receptor for a cytokine or hormone (e.g., bevacizumab (avastin), cetuximab (erbutux)). An anticancer drug can also be a drug under investigation for potential anti-cancer activity, such as those listed in Table 1. Anti-cancer drugs include monoclonal antibodies, such as but not limited to monoclonal antibodies that bind cytokines, hormones, or hormone receptors (e.g., antibodies that block activation of EGF or VEGF growth factors, such as Avastin, erbutux, herceptin), etc. The methods of the present invention include methods in which more than one additional anticancer drug is used in combination with 5-FU.

The method for decreasing the toxicity of a cancer drug treatment that includes administration of 5-FU or an analogue or prodrug thereof and an additional anticancer drug comprises administering 5,10-5,10-CH₂-THFA to the patient before, after, or concurrent with the administration of 5-FU (or an analogue or prodrug thereof). Preferably, administration of 5,10-CH₂-THFA is before administration of 5-FU. An additional anticancer drug can be administered before, after, or concurrent with administration of 5-FU.

Dosage for the one or more additional anticancer drugs used in a multidrug regimen of the present invention can also be determined by studies using escalating dosages and monitoring of toxicity and efficacy. In determining dosages of an anticancer drug to be used in combination therapy that have been used independently in chemotherapy regimens, practitioners can take into account dosages of drugs used in established chemotherapy regimens.

A number of chemotherapy protocols that combine 5-FU with one or more anticancer drugs (other than a folate cofactor of thymidylate synthase) are known in the field of cancer therapy. For example, anticancer protocols that include 5-FU in combination with one or more additional drugs (other than a folate cofactor) include but are not limited to therapies for breast cancer that include cyclophosphamide, epirubicin, and fluorouracil (see, for example, Levine M N, Bramwell V H, Pritchard K I et al. “Randomized trial of intensive cyclophosphamide, epirubicin, and fluorouracil chemotherapy compared with cyclophosphamide, methotrexate, and fluorouracil in premenopausal women with node-positive breast cancer.” J Clin Oncol 1998;16(8): 2651-8; herein incorporated by reference, particularly for disclosure of anticancer protocols the use 5-FU.) Anticancer protocols that include 5-FU in combination with one or more additional drugs (other than a folate cofactor) also include therapies for breast cancer that include cyclophosphamide, doxorubicin, and fluorouracil (see, for example, Bennett J M, Muss H B, Doroshaw J H, et al. “A randomized multicenter trial comparing mitoxantrone, cyclophophamide, and fluorouracil with doxorubicin, cyclophosphamide, and fluorouracil in the therapy of metastatic breast cancer.” J Clin Oncol 1988;6(10):1611-20; herein incorporated by reference, in particular for disclosure of anticancer protocols that include 5-FU.) The present invention includes the addition of 5,10-CH₂-THFA to chemotherapy regimens such as these to reduce the toxicity of the chemotherapy regimens.

Another example of an anticancer protocol to which 5,10-CH₂-THFA can be added to reduce the toxicity of treatment is a protocol for the treatment of head-and-neck cancer that includes the use of mitomycin C and fluorouracil as disclosed in Keane T J, Cummings B J, O'Sullivan B, Payne D, Rawlinson E, MacKenzie R, Danjoux C, Hodson I. “A randomized trial of radiation therapy compared to split course radiation therapy combined with mitomycin C and 5-fluorouracil as initial treatment for advanced laryngeal and hypopharyngeal squamous carcinoma.” IJ Radiation Oncology Biol Phys, 1993: 25(4): 613-8; herein incorporated by reference, in particular for disclosure relating to anticancer protocols that use 5-FU. In this case, the anticancer treatment protocol includes radiation therapy in addition to chemotherapy.

Yet other types of protocols to which 5,10-CH₂-THFA can be added to reduce the toxicity of treatment are anticancer protocols that combine 5-FU with mitomycin C, such as that disclosed in Keane T J, Cummings B J, O'Sullivan B, Payne D, Rawlinson E, MacKenzie R, Danjoux C, Hodson I. “A randomized trial of radiation therapy compared to split course radiation therapy combined with mitomycin C and 5-fluorouracil as initial treatment for advanced laryngeal and hypopharyngeal squamous carcinoma.” IJ Radiation Oncology Biol Phys, 1993:25(4):613-8; herein incorporated by reference, in particular for disclosure relating to anticancer protocols that include 5-FU, and others that combine the use of carboplatin with 5-FU as disclosed in Calais G, Alfonsi M, Bardet E, et al. “Randomized trial of radiation therapy versus concomitant chemotherapy and radiation therapy for advanced-stage oropharynx carcinoma.” J Natl Cancer Inst 1999; 91:2081-6, herein incorporated by reference, in particular for disclosure relating to anticancer protocols that include 5-FU. In these treatments, anticancer treatment protocols include radiation therapy in addition to chemotherapy.

The present invention includes methods of decreasing the toxicity of a protocol that includes analogs or prodrugs of 5-FU and an additional anticancer drug (other than a folate cofactor of thymidylate synthase) by co-administering 5,10-CH₂-THFA. Examples of anticancer regimens that include capecitabine and docetaxel are disclosed in O'Shaughnessy J, et al. Superior survival with capecitabine plus docetaxel combination therapy in anthracycline pre-treated patients with advanced breast cancer: phase III trial results. J Clin Oncol 2002;20:2812-23, herein incorporated by reference, particularly for disclosure of anticancer protocols using capecitabine. 5,10-CH₂-THFA can also be added to protocols that include tegafur-uracil (UFT) in combination with an additional cancer drug, for example, protocols that include oxaliplatin, as disclosed in Feliu J. et al. “Phase II study of UFT and oxaliplatin in first-line treatment of advanced colorectal cancer.” Br. J. Cancer 2004 91: 1758-62; herein incorporated by reference, particularly for disclosure of anticancer protocols using UFT.

The foregoing references to protocols are examples only, and are not intended to be limiting in any way. Anticancer protocols to which 5,10-CH₂-THFA can be added to reduce the toxicity of treatment can be obtained from any reputable source, including the scientific and medical literature, and the resources of hospitals, cancer centers, and clinics. It is within the scope of the invention to modify the dosages and schedules of either or both of 5-FU, 5,10-CH₂-THFA, and, where relevant, one or more additional anticancer drugs in reducing the toxicity of a protocol by including administration of 5,10-CH₂-THFA. Such modifications can be made by trained clinicians that monitor patient reaction to treatment according to accepted medical practices.

Some preferred embodiments of this aspect of the present invention include methods for reducing the toxicity of an anticancer drug regimen that includes 5-FU (or an analog or prodrug thereof) and a folate cofactor of thymidylate synthase in which 5,10-CH₂-THFA is substituted for leucovorin as the folate cofactor for thymidylate synthase. The methods comprise: obtaining an anticancer drug protocol that comprises 5-FU or an analogue or prodrug thereof and leucovorin; and substituting 5,10-CH₂-THFA for leucovorin in the anticancer drug protocol.

In some preferred embodiments of this aspect, the present invention includes methods for decreasing toxicity of an anticancer drug regimen that includes an analog or prodrug of 5-FU, such as, but not limited to, capecitabine or UFT, and leucovorin, where toxicity of the regimen is decreased by substituting 5,10-CH₂-THFA for leucovorin in the regimen.

In some preferred embodiments of this aspect, the present invention includes methods for decreasing the toxicity of an anticancer treatment that comprises 5-FU or an analog or prodrug of 5-FU, leucovorin, and at least one additional anticancer drug (other than 5-FU or a folate cofactor of thymidylate synthase) to a patient with cancer by substituting 5,10-5,10-CH₂-THFA for leucovorin in the drug regimen. The method comprises: obtaining an anticancer drug protocol that comprises 5-FU or an analogue or prodrug thereof, leucovorin; and at least one additional anticancer drug; and substituting 5,10-CH₂-THFA for leucovorin in the anticancer drug protocol.

Because of the anti-tumor activity and decreased systemic toxicity of 5,10-CH₂-THFA compared to leucovorin, and because of the similar chemical and metabolic pathways of leucovorin and 5,10-CH₂-THFA, the inventors contemplate that 5,10-CH₂-THFA can substitute for leucovorin in a range of current chemotherapy regimens. Nonlimiting examples of current drugs commonly used in combination with 5-FU plus leucovorin are Irinotecan (CPT-11), Oxaliplatin, gemcitabine, mitomycin C, levamisole, and vinorelbine. The present invention includes treatments that substitute 5,10-CH₂-THFA for leucovorin in these regimens. Substitution of 5,10-CH₂-THFA for leucovorin can provide equivalent or enhanced therapeutic effects with reduced toxicity. As nonlimiting examples, current drug combination regimens in which 5,10-CH₂-THFA can substitute for leucovorin include the following protocols used in the treatment of colorectal cancer:
AIO regimen (folinic acid, 5-FU, Irinotecan):
   Irinotecan (100 mg/m²) as a 2-hour infusion day 1; leucovorin (500 mg/m²) as a 2-hour infusion day 1; followed by 5-FU (2,000 mg/m²) intravenous (IV) bolus via ambulatory pump over 24 hours weekly×4 every 52 weeks.
Douillard regimen (folinic acid, 5-FU, Irinotecan):
   Irinotecan (180 mg/m²) as a 2-hour infusion day 1; leucovorin (200 mg/m²) as a 2-hour infusion days 1 and 2; followed by a loading dose of 5-FU (400 mg/m²) IV bolus, then 5-FU (600 mg/m²) via ambulatory pump over 22 hours days 1 and 2 every 2 weeks.
FOLFOX4 regimen (oxaliplatin, leucovorin, 5-FU):
   Oxaliplatin (85 mg/m²) as a 2-hour infusion day 1; leucovorin (200 mg/m²) as a 2-hour infusion days 1 and 2; followed by a loading dose of 5-FU (400 mg/m²) IV bolus, then 5-FU (600 mg/m²) via ambulatory pump over 22 hours days 1 and 2 every 2 weeks.
FOLFOX6 regimen (oxaliplatin, leucovorin, 5-FU):
   Oxaliplatin (85-100 mg/m²) as a 2-hour infusion day 1; leucovorin (400 mg/m²) as a 2-hour infusion day 1; followed by a loading dose of 5-FU (400 mg/m²) IV bolus on day 1, then 5-FU (2,400-3,000 mg/m²) via ambulatory pump over 46 hours every 2 weeks.
FOLFIRI regimen (folinic acid, 5-FU, Irinotecan):
   Irinotecan (180 mg/m²) as a 2-hour infusion day 1; leucovorin (400 mg/m²) as a 2-hour infusion day 1; followed by a loading dose of 5-FU (400 mg/m²) IV bolus on day 1, then 5-FU (2,400-3,000 mg/m²) via ambulatory pump over 46 hours every 2 weeks.
IFL (or Saltz) regimen (Irinotecan, 5-FU, leucovorin):
   Irinotecan (125 mg/m²), 5-FU (500 mg/m²) IV bolus, and leucovorin (20 mg/M²) IV bolus weekly for 4 out of 6 weeks.

Other regimens in which 5,10-CH₂-THFA can substitute for leucovrin include in combination with 5-FU and at least one other anticancer drug include, for example, FOLFUGEM 1 ((leucovorin 400 mg/m2 combined with 5-flurorouracil (FU) bolus 400 mg/m2 then 5-FU 2-3 g/m2/46 hours and gemcitabine 1000 mg/m2 in 30 min) and FOLFUGEM 2 (leucovorin 400 mg/m2 in 2 hours followed by 5-FU 1000 mg/m2 in 22 hours, then gemcitabine 800 mg/m2 (10 mg/m2/min) with cycles every 14 days) used to treat pancreatic cancer (as disclosed in Andre et al. “Phase II study of leucovorin, 5-fluorouracil, and gemcitabine for locally advanced and metastatic pancreatic cancer (FOLFUGEM 2) Gastroeneterol Clin Biol ; 2004 28: 645-650, herein incorporated by reference, in particular for disclosure of cancer treatment protocols that include 5-FU.)

In another example, 5,10-CH₂-THFA can substitute for leucovorin in combination therapies that also include 5-FU and levamisole (as disclosed in Poplin et al. “Phase III Southwest Oncology Group 9415/Intergroup 0153 randomized trial of fluorouracil, leucovorin, and levamisole versus fluorouracil continuous infusion and levamisole for adjuvant treatment of stage III and high-risk stage II colon cancer.” J. Clin Oncol. 2005 23: 1819-25; herein incorporated by reference, in particular for disclosure of cancer treatment protocols that include 5-FU.).

In yet another example, 5,10-CH₂-THFA can substitute for leucovorin in combination therapies that also include 5-FU and vinorelbine (as disclosed in Yeh et al. “Phase II study of weekly vinorelbine and 24-hr infusion of high-dose 5-fluorouracil plus leucovorin as first-line treatment of advanced breast cancer.” Br. J. Cancer 2005 92: 1013-8; herein incorporated by reference, in particular for disclosure of cancer treatment protocols that include 5-FU.).

The forgoing examples are not intended to be limiting in any way. For example, dosages and regimens can be altered or optimized to minimize toxicity to the patient or improve efficacy. In addition, many anti-cancer drugs that are not described herein can be combined with 5,10-CH₂-THFA and 5-FU. 5,10-CH₂-THFA can also be substituted for leucovorin in protocols in which 5-FU and leucovorin are used in combination with more than one additional anticancer drug. We also include 5,10-CH₂-THFA use in combination therapies with next-generation forms of 5-FU, specifically oral forms of 5-FU (e.g. Xeloda, capecitabine) and UFT.

Other uses of 5,10-CH₂-THFA are in combination therapy with new classes of biologic anti-tumor reagents, such as monoclonal antibodies with anti-tumor activity. Examples of antibodies that might be combined with 5,10-CH₂-THFA (preferably with 5-FU) include anti-VEGF antibody (e.g. bevacuzimab or “Avastin”) and anti-EGF receptor (e.g. Erbitux, cetuximab, herceptin). As shown in Examples 1 and 2, combination 5-FU/5,10-CH₂-THFA /Avastin treatment of colorectal carcinoma in nude mice inhibits tumor growth more than the other drug combinations.

Because of the lower toxicity profile of 5,10-CH₂-THFA disclosed herein, the present invention also includes 5,10-CH₂-THFA use in combination with drugs that typically are considered too toxic for widespread use. For example, 5-FU/5, 10-CH₂-THFA/Cisplatin therapy is such a combination. Cisplatin, a platinum-based chemotherapy agent is highly toxic. In addition, the lower toxicity profile of 5,10-CH₂-THFA can allow use of either increased concentrations of drugs (e.g. 5-FU) or prolonged dosing periods. In turn this may increase drug efficacy.

The present invention also includes the use of 5,10-CH₂-THFA in place of leucovorin (leucovorin) in therapies that do not use 5-FU. For example, based on the lower toxicity profile and increased activity of 5,10-CH₂-THFA compared to leucovorin (leucovorin), 5,10-CH₂-THFA can be used for methotrexate rescue therapy. This mode of therapy currently uses leucovorin.

V. Methods for Increasing the Efficacy of An Anticancer Drug Treatment Regime That Includes 5-FU

The present invention also provides methods for increasing the efficacy of an anticancer drug treatment regimen that includes administration of 5-5-FU or an analog or prodrug of 5-FU to a cancer patient by co-administering 5,10-CH₂-THFA.

In one aspect, the method comprises: obtaining an anticancer drug regimen that comprises 5-fluorouracil or an analogue or prodrug thereof, and adding 5,10-CH₂-THFA to the drug regimen to increase the efficacy of the anticancer drug regimen. In preferred embodiments of this aspect of the present invention, 5-FU and 5,10-CH₂-THFA are administered to the patient in the absence of leucovorin (folinic acid, FA). The method for increasing the efficacy of a cancer drug treatment that includes administration of 5-FU or an analogue or prodrug thereof comprises administering 5,10-CH₂-THFA to the patient before, after, or concurrent with the administration of 5-FU to reduce the toxicity of 5-FU. Preferably, administration of 5,10-CH₂-THFA is before administration of 5-FU.

In a related aspect, the invention provides methods for increasing survivorship of a cancer patient by adding 5,10-CH₂-THFA to an anticancer drug regimen administered to the patient that includes 5-FU or an analog or prodrug of 5-FU. The method comprises: obtaining an anticancer drug protocol that comprises 5-fluorouracil or an analogue or prodrug thereof, adding 5,10-CH₂-THFA to the anticancer drug protocol; and treating a cancer patient with the modified anticancer drug protocol. The method includes administering 5,10-CH₂-THFA to the patient before, after, or concurrent with the administration of 5-FU. In preferred embodiments of this aspect of the present invention, 5-FU and 5,10-CH₂-THFA are administered to the patient in the absence of leucovorin (folinic acid, FA).

A cancer patient can be a patient with any type of cancer. In some preferred embodiments of the present invention in which 5,10-CH₂-THFA is administered to a cancer patient receiving 5-FU, the patient has a tumor type that is currently treated with 5-FU, such as, for example, colorectal carcinoma, pancreatic cancer, breast cancer, head-and-neck cancer, or stomach cancer.

Efficacy of an anticancer drug regimen can be determined by methods such as but not limited to: tumor size after treatment, the rate of tumor growth (or shrinkage), detection of cancer cells or markers, the length of remission after treatment, and the survivorship of the cancer patients treated with the regimen.

Those skilled in the art of cancer treatment and chemotherapy would be able to determine optimal dosages and regimens for 5,10-CH₂-THFA and 5-FU using well-established protocols for evaluating toxicity and efficacy. Some preferred treatments of cancer patients with 5-FU and 5,10-CH₂-THFA are regimens using from 10 milligrams to 1 gram of 5,10-CH₂-THFA per m², preferably from 20 milligrams to 500 milligrams of 5,10-CH₂-THFA per m², and more preferably from about 30 milligrams to about 250 milligrams of 5,10-CH₂-THFA per m². For example, a preferred dose of 5,10-CH₂-THFA can be from about 30 to about 120 milligrams per m². The foregoing are general guidelines only that can be expanded or altered based on for example, cancer type and grade, patient age, health status, and sex, the particular drugs used in combination, the route and frequency of administration, and experimental and clinical findings using a multidrug combination.

Dosage of 5-FU can be from about 25 milligrams to about 5 grams per m², and is preferably from about 50 milligrams to 2.5 grams per m², and more preferably from about 100 milligrams to about 1 gram per m². For example, a preferred dose of 5-FU can be from about 250 to about 700 milligrams per m². The foregoing are general guidelines only that can be expanded or altered based on for example, cancer type and grade, patient age, health status, and sex, the particular drugs used in combination, the route and frequency of administration, and experimental and clinical findings using a multidrug combination. 5-FU can be administered by any feasible means, including injection or IV feed.

In some preferred embodiments, a prodrug or analog of 5-FU is used in combination therapy rather than 5-FU itself. In the tissues of a patient, 5-FU is converted to 5-fluoro-2′-deoxyuridylate (FdUMP) the inhibitor of thymidylate synthase. In the present application, “analog or prodrug of 5-FU” is used to mean an analog or prodrug that can be directly or indirectly converted to an inhibitor of thymidylate synthase, such as FdUMP. One prodrug of 5-FU that can be used in the methods of the present invention is N4-pentoxylcarbonyl-5′-deoxy-5-fluorocytidine (capecitabine). In one preferred embodiment, the method of the present invention comprises administering N4-pentoxylcarbonyl-5′-deoxy-5-fluorocytidine (capecitabine); 5,10-CH₂-THFA; and at least one additional anticancer drug to a patient with cancer. The dosage of capecitabine can be determined by skilled clinicians and depends in part on the frequency of administration. For example, the of daily dosage of capecitabine can be from about 500 mg to about 7500 mg per m², preferably from about 1000 mg to about 5000 mgs per m², and more preferably from about 1500 mg to about 3000 mg per m². The dose can be divided into one to six (preferably two) administrations per day. The foregoing are general guidelines only that can be expanded or altered based on for example, cancer type and grade, patient age, health status, and sex, the particular drugs used in combination, the route and frequency of administration, and experimental and clinical findings using a multidrug combination. Capecitabine can be administered by any feasible means including injection, IV feed, or in an oral formulation.

An analog combination that can be used in the methods of the present invention is Tegafur (TF) and uracil (U) used in a 1:4 combination known as UFT. In one preferred embodiment, the method of the present invention comprises administering UFT; 5,10-CH₂-THFA; and at least one additional anticancer drug to a patient with cancer. The dosage of UFT can be determined by skilled clinicians and depends in part on the frequency of administration. For example, the daily dosage of UFT can be from about 50 mg to about 3000 mg per m², preferably from about 100 mg to about 2000 mg per m², and more preferably from about 200 mg to about 1000 mg per m². Anticancer regimens that include UFT can optionally also include calcium folinate administered with UFT. The foregoing are general guidelines only that can be expanded or altered based on for example, cancer type and grade, patient age, health status, and sex, the particular drugs used in combination, the route and frequency of administration, and experimental and clinical findings using a multidrug combination. UFT can be administered by any feasible means, including injection, IV feed, or in an oral formulation.

Some examples of anticancer drug protocols that use capecitabine are described in Blum JL, et al. “Multicenter phase II study of capecitabine in paclitaxel-refractory metastatic breast cancer.” J Clin Oncol 1999; 17:485-93; in Hoff et al. “Comparison of oral capecitabine versus intravenous fluorouracil plus leucovorin as first-line treatment in 605 patients with metastatic colorectal cancer: results of a randomized phase III study.” J Clin Oncol 2001;19(8):2282-92; and in Van Cutsem E, et al. “Oral capecitabine compared with intravenous fluorouracil plus leucovorin in patients with metastatic colorectal cancer: results of a large phase III study.” J Clin Oncol 2001; 19(21):4097-106; all of which are herein incorporated by reference, in particular for disclosure of chemotherapy regimens using capecitabine. The present invention includes administering 5,10-CH₂-THFA in protocols that include capecitabine to improve efficacy of treatment.

For example, one protocol includes administering capecitabine (1000-1250 mg per m²) twice daily for two weeks, followed by a one week rest period, and then followed by further three week cycles. 5,10-CH₂-THFA can be added to protocols such as these, for example, and the protocols can be optimized based on clinical trials for toxicity and efficacy.

In other preferred embodiments of this aspect, the present invention includes methods for increasing the efficacy of an anticancer treatment that comprises administering 5-FU or an analog or prodrug of 5-FU and at least one additional anticancer drug (other than 5-FU or a folate cofactor of thymidylate synthase) to a patient with cancer by co-administering 5,10-5,10-CH₂-THFA. The method includes: obtaining an anticancer drug protocol that comprises 5-fluorouracil or an analogue or prodrug thereof and at least one additional anticancer drug, and adding 5,10-methylene tetrahydrofolate to the anticancer drug protocol to obtain an anticancer drug protocol having increased efficacy.

The method for increasing the efficacy of a cancer drug treatment that includes administration of 5-FU or an analogue or prodrug thereof and an additional anticancer drug comprises administering 5,10-5,10-CH₂-THFA to the patient before, after, or concurrent with the administration of 5-FU (or an analogue or prodrug thereof). Preferably, administration of 5,10-CH₂-THFA is before administration of 5-FU. An additional anticancer drug can be administered before, after, or concurrent with administration of 5-FU.

In a related aspect, the invention provides methods for increasing survivorship of a cancer patient by adding 5,10-5,10-CH₂-THFA to an anticancer drug regimen administered to the patient that includes 5-5-FU or an analog or prodrug of 5-FU, and at least one additional anticancer drug (other than 5-FU or a folate cofactor of thymidylate synthase). The method comprises: obtaining an anticancer drug protocol that comprises 5-FU or an analogue or prodrug thereof and at least one additional anticancer drug; adding 5,10-CH₂-THFA to the anticancer drug protocol; and treating a cancer patient with the modified anticancer drug protocol. The method includes administering 5,10-CH₂-THFA to the patient before, after, or concurrent with the administration of 5-FU. In preferred embodiments of this aspect of the present invention, 5-FU and 5,10-CH₂-THFA are administered to the patient in the absence of leucovorin (folinic acid, FA). An additional anticancer drug can be administered before, after, or concurrent with administration of 5-FU.

Dosage for the one or more additional anticancer drugs used in a multidrug regimen of the present invention can also be determined by studies using escalating dosages and monitoring of toxicity and efficacy. In determining dosages of an anticancer drug to be used in combination therapy that have been used independently in chemotherapy regimens, practitioners can take into account dosages of drugs used in established chemotherapy regimens.

A number of chemotherapy protocols that combine 5-FU with one or more anticancer drugs (other that folate cofactors of thymidylate synthase) are known in the field of cancer therapy. For example, anticancer protocols that include 5-FU in combination with one or more additional drugs (other than a folate cofactor of thymidylate synthase) include but are not limited to therapies for breast cancer that include cyclophosphamide, epirubicin, and fluorouracil (see, for example, Levine M N, Bramwell V H, Pritchard K I et al. “Randomized trial of intensive cyclophosphamide, epirubicin, and fluorouracil chemotherapy compared with cyclophosphamide, methotrexate, and fluorouracil in premenopausal women with node-positive breast cancer.” J Clin Oncol 1998;16(8): 2651-8; herein incorporated by reference, particularly for disclosure of anticancer protocols that use 5-FU.) Anticancer protocols that include 5-FU in combination with one or more additional drugs (other than a folate cofactor) also include therapies for breast cancer that include cyclophosphamide, doxorubicin, and fluorouracil (see, for example, Bennett J M, Muss H B, Doroshaw J H, et al. “A randomized multicenter trial comparing mitoxantrone, cyclophophamide, and fluorouracil with doxorubicin, cyclophosphamide, and fluorouracil in the therapy of metastatic breast cancer.” J Clin Oncol 1988;6(10):1611-20; herein incorporated by reference, in particular for disclosure of anticancer protocols that include 5-FU.). The addition of 5,10-CH₂-THFA can enhance the efficacy of these chemotherapy regimens and improve survivorship of patient treated with the modified regimens.

Another example of an anticancer protocol to which 5,10-CH₂-THFA can be added to increase the efficacy of treatment is a protocol for the treatment of head-and-neck cancer that includes the use of mitomycin C and fluorouracil as disclosed in Keane T J, Cummings B J, O'Sullivan B, Payne D, Rawlinson E, MacKenzie R, Danjoux C, Hodson I. “A randomized trial of radiation therapy compared to split course radiation therapy combined with mitomycin C and 5-fluorouracil as initial treatment for advanced laryngeal and hypopharyngeal squamous carcinoma.” IJ Radiation Oncology Biol Phys, 1993: 25(4): 613-8; herein incorporated by reference, particularly for disclosure relating to anticancer protocols that use 5-FU. In this case, the anticancer treatment protocol includes radiation therapy in addition to chemotherapy.

Yet other types of protocols to which 5,10-CH₂-THFA can be added to increase the efficacy of treatment are anticancer protocols that combine 5-FU with mitomycin C, such as that disclosed in Keane T J, Cummings B J, O'Sullivan B, Payne D, Rawlinson E, MacKenzie R, Danjoux C, Hodson I. “A randomized trial of radiation therapy compared to split course radiation therapy combined with mitomycin C and 5-fluorouracil as initial treatment for advanced laryngeal and hypopharyngeal squamous carcinoma.” IJ Radiation Oncology Biol Phys, 1993:25(4):613-8; herein incorporated by reference, particularly for disclosure relating to anticancer protocols that include 5-FU, and others that combine the use of carboplatin with 5-FU as disclosed in Calais G, Alfonsi M, Bardet E, et al. “Randomized trial of radiation therapy versus concomitant chemotherapy and radiation therapy for advanced-stage oropharynx carcinoma.” J Natl Cancer Inst 1999; 91:2081-6, herein incorporated by reference, particulary for disclosure relating to anticancer regimens that use 5-FU. In these treatments, anticancer treatment protocols include radiation therapy in addition to chemotherapy.

The present invention includes methods of increasing the efficacy of a protocol that includes analogs or prodrugs of 5-FU and an additional anticancer drug (other than a folate cofactor of thymidylate synthase) by co-administering 5,10-CH₂-THFA. Examples of anticancer regimens that include capecitabine and docetaxel are disclosed in O'Shaughnessy J, et al. Superior survival with capecitabine plus docetaxel combination therapy in anthracycline pre-treated patients with advanced breast cancer: phase III trial results. J Clin Oncol 2002;20:28 12-23, herein incorporated by reference, particularly for disclosure of anticancer protocols using capecitabine. 5,10-CH₂-THFA can also be added to protocols that include tegafur-uracil (UFT) in combination with an additional cancer drug, for example, protocols that include oxaliplatin, as disclosed in Feliu J. et al. “Phase II study of UFT and oxaliplatin in first-line treatment of advanced colorectal cancer.” Br. J. Cancer 2004 91: 1758-62; herein incorporated by reference, particularly for disclosure of anticancer protocols using UFT.

The foregoing references to protocols are examples only, and are not intended to limit the invention in any way. Anticancer protocols to which 5,10-CH₂-THFA can be added to increase the efficacy of treatment can be obtained from any reputable source, including the scientific and medical literature, and the resources of hospitals, cancer centers, and clinics. It is within the scope of the invention to modify the dosages and schedules of either or both of 5-FU, 5,10-CH₂-THFA, and, where relevant, one or more additional anticancer drugs in increasing the efficacy of a protocol by including administration of 5,10-CH₂-THFA. Such modifications can be made by trained clinicians that monitor patient response to treatment according to accepted medical practices.

In some preferred embodiments, the present invention includes methods of increasing the efficacy of an anticancer drug regimen that includes 5-FU and a folate cofactor of thymidylate synthase in which 5,10-CH₂-THFA is substituted for leucovorin as the thymidylate synthase cofactor. The invention includes methods of increasing the efficacy of an anticancer drug regimen, in which the anticancer drug regimen includes 5-FU and a folate cofactor of thymidylate synthase and efficacy is increased by substituting 5,10-CH₂-THFA for leucovorin as the thymidylate synthase cofactor.

In preferred embodiments of this aspect of the present invention, the method comprises: obtaining an anticancer drug regimen that comprises 5-FU or an analogue or prodrug thereof, leucovorin, and an additional anticancer drug; and substituting 5,10-CH₂-THFA for leucovorin in the drug regimen to obtain a drug regimen with improved efficacy.

In a related aspect, the invention provides methods for increasing survivorship of a cancer patient by substituting 5,10-5,10-CH₂-THFA for leucovorin in an anticancer drug regimen administered to the patient that includes 5-FU or an analog or prodrug of 5-FU. The method comprises: obtaining an anticancer drug protocol that comprises 5-FU or an analogue or prodrug thereof and leucovorin; substituting 5,10-CH₂-THFA for leucovorin in the anticancer drug protocol; and treating a cancer patient with the modified anticancer drug protocol. The method includes administering 5,10-CH₂-THFA to the patient before, after, or concurrent with the administration of 5-FU.

In some preferred embodiments of this aspect, the present invention includes methods for increasing the efficacy of an anticancer drug regimen that includes an analog or prodrug of 5-FU, such as, but not limited to, capecitabine or UFT, and leucovorin, where efficacy of the regimen is increased by substituting 5,10-CH₂-THFA for leucovorin in the regimen. The present invention also provides methods for increasing survivorship of a cancer patient by substituting 5,10-CH₂-THFA for leucovorin in an anticancer drug regimen administered to the patient that includes an analog or prodrug of 5-FU, such as but not limited to capecitabine or UFT.

In some preferred embodiments of this aspect, the present invention includes methods for increasing the efficacy of an anticancer treatment that comprises 5-FU or an analog or prodrug of 5-FU, leucovorin, and at least one additional anticancer drug (other than 5-FU or a folate cofactor of thymidylate synthase) to a patient with cancer by substituting 5,10-5,10-CH₂-THFA for leucovorin in the drug regimen. The method comprises: obtaining an anticancer drug protocol that comprises 5-FU or an analogue or prodrug thereof; leucovorin; and at least one additional anticancer drug; and substituting 5,10-CH₂-THFA for leucovorin in the anticancer drug protocol.

In a related aspect, the invention provides methods for increasing survivorship of a cancer patient by substituting 5,10-5,10-CH₂-THFA for leucovorin in an anticancer drug regimen administered to the patient that includes 5-FU or an analog or prodrug of 5-FU, and at least one additional anticancer drug (other than 5-FU or a folate cofactor of thymidylate synthase). The method comprises: obtaining an anticancer drug protocol that comprises 5-FU or an analogue or prodrug thereof, leucovorin, and at least one additional anticancer drug; substituting 5,10-CH₂-THFA for leucovorin in the anticancer drug protocol; and treating a cancer patient with the modified anticancer drug protocol. The method includes administering 5,10-CH₂-THFA to the patient before, after, or concurrent with the administration of 5-FU. An additional anticancer drug can be administered before, after, or concurrent with administration of 5-FU.

Because of the anti-tumor activity and decreased systemic toxicity of 5,10-CH₂-THFA compared to leucovorin, and because of the similar chemical and metabolic pathways of leucovorin and 5,10-CH₂-THFA, the inventors contemplate that 5,10-CH₂-THFA can substitute for leucovorin in a range of current chemotherapy regimens. Examples of current drugs commonly used in combination with 5-FU plus leucovorin are Irinotecan (CPT-11), Oxaliplatin, gemcitabine, levamisole, mitomycin C, and vinorelbine. The present invention includes treatments that substitute 5,10-CH₂-THFA for leucovorin in these regimens. Substitution of 5,10-CH₂-THFA for leucovorin can provide enhanced therapeutic effects with reduced toxicity. As nonlimiting examples, current drug combination regiments that 5,10-CH₂-THFA can substitute for leucovorin include:
AIO regimen (folic acid, 5-FU, Irinotecan):
   Irinotecan (100 mg/m²) as a 2-hour infusion day 1; leucovorin (500 mg/m²) as a 2-hour infusion day 1; followed by 5-FU (2,000 mg/m²) intravenous (IV) bolus via ambulatory pump over 24 hours weekly×4 every 52 weeks.
Douillard regimen (folic acid, 5-FU, Irinotecan):
   Irinotecan (180 mg/m²) as a 2-hour infusion day 1; leucovorin (200 mg/m²) as a 2-hour infusion days 1 and 2; followed by a loading dose of 5-FU (400 mg/m²) IV bolus, then 5-FU (600 mg/m²) via ambulatory pump over 22 hours days 1 and 2 every 2 weeks.
FOLFOX4 regimen (oxaliplatin, leucovorin, 5-FU):
   Oxaliplatin (85 mg/m²) as a 2-hour infusion day 1; leucovorin (200 mg/m²) as a 2-hour infusion days 1 and 2; followed by a loading dose of 5-FU (400 mg/m²) IV bolus, then 5-FU (600 mg/m²) via ambulatory pump over 22 hours days 1 and 2 every 2 weeks.
FOLFOX6 regimen (oxaliplatin, leucovorin, 5-FU):
   Oxaliplatin (85-100 mg/M²) as a 2-hour infusion day 1; leucovorin (400 mg/m²) as a 2-hour infusion day 1; followed by a loading dose of 5-FU (400 mg/m²) IV bolus on day 1, then 5-FU (2,400-3,000 mg/m²) via ambulatory pump over 46 hours every 2 weeks.
FOLFIRI regimen (folic acid, 5-FU, Irinotecan):
   Irinotecan (180 mg/M²) as a 2-hour infusion day 1; leucovorin (400 mg/m²) as a 2-hour infusion day 1; followed by a loading dose of 5-FU (400 mg/M²) IV bolus on day 1, then 5-FU (2,400-3,000 mg/m²) via ambulatory pump over 46 hours every 2 weeks.
IFL (or Saltz) regimen (Irinotecan, 5-FU, leucovorin):
   Irinotecan (125 mg/m²), 5-FU (500 mg/m²) IV bolus, and leucovorin (20 mg/m²) IV bolus weekly for 4 out of 6 weeks.

Other regimens in which 5,10-CH₂-THFA can substitute for leucovrin include in combination with 5-FU and at least one other anticancer drug include, for example, FOLFUGEM 1 ((leucovorin 400 mg/M² combined with 5-flurorouracil (FU) bolus 400 mg/m² then 5-FU 2-3 g/m²/46 hours and gemcitabine 1000 mg/m² in 30 min) and FOLFUGEM 2 (leucovorin 400 mg/m² in 2 hours followed by 5-FU 1000 mg/m² in 22 hours, then gemcitabine 800 mg/m² (10 mg/m²/min) with cycles every 14 days) used to treat pancreatic cancer (as disclosed in Andre et al. “Phase II study of leucovorin, 5-fluorouracil, and gemcitabine for locally advanced and metastatic pancreatic cancer (FOLFUGEM 2) Gastroeneterol Clin Biol ; 2004 28: 645-650, herein incorporated by reference, in particular for disclosure of cancer treatment protocols that include 5-FU.)

In another example, 5,10-CH₂-THFA can substitute for leucovorin in combination therapies that also include 5-FU and levamisole (as disclosed in Poplin et al. “Phase III Southwest Oncology Group 9415/Intergroup 0153 randomized trila of fluorouracil, leucovorin, and levamisole versus fluorouracil continuous infusion and levamisole for adjuvant treatment of stage III and high-risk stage II colon cancer.” J. Clin Oncol. 2005 23: 1819-25; herein incorporated by reference, in particular for disclosure of cancer treatment protocols that use 5-FU.).

In yet another example, 5,10-CH₂-THFA can substitute for leucovorin in combination therapies that also include 5-FU and vinorelbine (as disclosed in Yeh et al. “Phase II study of weekly vinorelbine and 24-hr infusion of high-dose 5-fluorouracil plus leucovorin as first-line treatment of advanced breast cancer.” Br. J. Cancer 2005 92: 1013-8; herein incorporated by reference, in particular for disclosure of cancer treatment protocols that include 5-FU.).

The forgoing examples are not intended to be limiting in any way. For example, dosages and regimens can be altered or optimized to minimize toxicity to the patient or improve efficacy. In addition, many anti-cancer drugs that are not described herein can be combined with 5,10-CH₂-THFA and 5-FU. We also propose 5,10-CH₂-THFA use in combination therapies with next-generation forms of 5-FU, specifically oral forms of 5-FU (e.g. Xeloda, capecitabine) and UFT.

Other uses of 5,10-CH₂-THFA are in combination therapy with new classes of biologic anti-tumor reagents, such as monoclonal antibodies with anti-tumor activity. Examples of antibodies that might be combined with 5,10-CH₂-THFA (preferably with 5-FU) include anti-VEGF antibody (e.g. Avastin, Bevacuzimab) and anti-EGF receptor (e.g. Erbitux, cetuximab, herceptin). As shown in Examples 1 and 2, combination 5-FU/5,10-CH₂-THFA /Avastin treatment of colorectal carcinoma in nude mice inhibits tumor growth more than the other drug combinations.

In other aspects of methods in which 5,10-CH₂-THFA is added to a treatment regimen that includes 5-FU (or an analog or prodrug thereof) and an additional anti-cancer drug, the inventors contemplate that at least one of the one or more additional anti-cancer drugs can be administered at an increased dosage relative to the dosage typically used for the additional anti-cancer drug in a regimen that includes 5-FU. Thus, the invention includes a method of increasing the efficacy of an anticancer drug protocol that includes 5-FU and at least one additional anticancer drug (other than 5-FU or an analog or prodrug thereof, or a folate cofactor of thymidylate synthase), by adding 5,10-CH₂-THFA to the drug regimen and increasing the dosage of at least one of the one or more additional anticancer drugs. The method includes: obtaining an anticancer drug protocol that includes 5-FU or an analog or prodrug of 5-FU and at least one additional anticancer drug (other than 5-FU or an analog or prodrug of 5-FU or a folate cofactor of thymidylate synthase); adding 5,10-CH₂-THFA to the anticancer drug protocol; and increasing the dosage of the one or more additional anticancer drugs in the anticancer drug protocol. In this aspect, adding 5,10-CH₂-THFA to the anticancer regimen while increasing the dosage of an additional anticancer drug used in the regimen can increase the efficacy of a treatment without prohibitively increasing toxicity.

In a related aspect, the invention includes methods of increasing the survivorship of a cancer patient by adding 5,10-CH₂-THFA to an anticancer regimen that includes 5-FU and one or more additional anticancer drugs (other than 5-FU or an analog or prodrug of 5-FU or a folate cofactor of thymidylate synthase) and increasing the dosage of at least one of the one or more additional anticancer drugs used in the regimen.

A number of chemotherapy protocols that combine 5-FU with one or more anticancer drugs (other that folate cofactors of thymidylate synthase) are known in the field of cancer therapy. For example, protocols referenced in this application include protocols in which 5-FU is combined with cyclophosphamide, epirubicin, docorubicin, carboplatin, or mitomycin C. These examples are in no way limiting to the scope of the invention. Other protocols known or used in the future in the field of cancer therapy that use these or other anti-cancer drugs in combination with 5-FU can also be modified by including 5,10-CH₂-THFA and increasing the dosage of at least one of the one or more additional anticancer drugs.

The present invention includes methods of increasing the efficacy of a protocol that includes analogs or prodrugs of 5-FU and at least one additional anticancer drug (other than a folate cofactor of thymidylate synthase) by co-administering 5,10-CH₂-THFA. An anticancer regimen that includes capecitabine and docetaxel, and an anticancer regimen that includes UFT and oxaliplatin, are referenced herein as nonlimiting examples of protocols that can be modified including 5,10-CH₂-THFA and increasing the dosage of the additional anticancer drug.

The foregoing references to protocols are examples only, and are not intended to limit the invention in any way. Anticancer protocols to which 5,10-CH₂-THFA can be added can be obtained from any reputable source, including the scientific and medical literature, and the resources of hospitals, cancer centers, and clinics. Dose escalation studies can be performed according to established protocols that monitor toxicity and efficacy. It is within the scope of the invention to modify the dosages and schedules of either or both of 5-FU, 5,10-CH₂-THFA, as well as one or more additional anticancer drugs, in optimizing anticancer treatment protocols. Such modifications can be made by trained clinicians that monitor patient response to treatment according to accepted medical practices.

In yet another related aspect, the invention provides a method of increasing the efficacy of an anticancer drug protocol of an anticancer drug protocol that comprises 5-FU or an analog or prodrug of 5-FU, leucovorin, and at least one additional anticancer drug by replacing leucovorin with 5,10-CH₂-THFA in the protocol and increasing the dosage of at least one additional anticancer drug. The method includes: obtaining an anticancer drug protocol that includes 5-FU or an analog or prodrug of 5-FU, leucovorin, and at least one additional anticancer drug (other than 5-FU or an analog or prodrug of 5-FU or a folate cofactor of thymidylate synthase); substituting 5,10-CH₂-THFA for leucovorin in the anticancer drug protocol; and increasing the dosage of the at least one additional anticancer drug in the anticancer drug protocol. In this aspect, substituting 5,10-CH₂-THFA for leucovorin in the anticancer while increasing the dosage of an additional anticancer drug used in the regimen can increase the efficacy of a treatment without prohibitively increasing toxicity.

In a related aspect, the invention includes methods of increasing the survivorship of a cancer patient by substituting 5,10-CH₂-THFA for leucovorin in an anticancer regimen that includes 5-FU and one or more additional anticancer drugs (other than 5-FU or an analog or prodrug of 5-FU or a folate cofactor of thymidylate synthase) and increasing the dosage of at least one of the one or more additional anticancer drugs used in the regimen.

A number of chemotherapy protocols that combine 5-FU and leucovorin with one or more anticancer drugs (other that folate cofactors of thymidylate synthase) are known in the field of cancer therapy. For example, protocols referenced in this application include protocols in which 5-FU is combined gemcitabine, vinorelbine, levamisole, irinotecan, oxaliplatin, or mitomycin C. These examples are in no way limiting to the scope of the invention. Other protocols known or used in the future in the field of cancer therapy that use these or other anti-cancer drugs in combination with 5-FU and leucovorin can also be modified by substituting 5,10-CH₂-THFA for leucovorin and increasing the dosage of at least one of the one or more additional anticancer drugs.

The present invention includes methods of increasing the efficacy of a protocol that includes analogs or prodrugs of 5-FU, leucovorin, and at least one additional anticancer drug (other than a folate cofactor of thymidylate synthase) by substituting 5,10-CH₂-THFA and increasing the dosage of an additional anticancer drug.

The foregoing references to protocols are examples only, and are not intended to limit the invention in any way. Anticancer protocols comprising multiple anticancer drugs to which 5,10-CH₂-THFA can be substituted for leucovorin can be obtained from any reputable source, including the scientific and medical literature, and the resources of hospitals, cancer centers, and clinics. Dose escalation studies can be performed according to established protocols that monitor toxicity and efficacy. It is within the scope of the invention to modify the dosages and schedules of either or both of 5-FU, 5,10-CH₂-THFA, as well as one or more additional anticancer drugs, in optimizing anticancer treatment protocols. Such modifications can be made by trained clinicians that monitor patient response to treatment according to accepted medical practices.

The inventors also contemplate that 5-FU can be administered at an increased dosage relative to the dosage typically used in combination therapy when 5,10-CH₂-THFA is added to the drug regimen. Thus, the invention includes a method of increasing the efficacy of an anticancer drug protocol by increasing the dosage of 5-FU used in a drug regimen for treating cancer that includes 5-FU (or an analog or prodrug thereof) and an additional anticancer drug (other than a folate cofactor of thymidylate synthase) by adding 5,10-CH₂-THFA to the drug regimen. The method includes: obtaining an anticancer drug protocol that includes 5-FU or an analog or prodrug of 5-FU and at least one additional anticancer drug (other than a folate cofactor of thymidylate synthase); adding 5,10-CH₂-THFA to the anticancer drug protocol; and increasing the dosage of 5-FU in the anticancer drug protocol. In this aspect, adding 5,10-CH₂-THFA to the anticancer regimen while increasing the dosage of 5-FU used in the regimen can increase the efficacy of a treatment without prohibitively increasing toxicity.

In yet another related aspect, the invention provides a method of increasing the dose of 5-FU in an anticancer drug protocol that comprises 5-FU or an analog or prodrug of 5-FU, leucovorin, and an additional anticancer drug by replacing leucovorin with 5,10-CH₂-THFA. The method includes: obtaining an anticancer drug protocol that includes 5-FU or an analog or prodrug of 5-FU, leucovorin, and at least one additional anticancer drug (other than a folate cofactor of thymidylate synthase); substituting 5,10-CH₂-THFA for leucovorin in the anticancer drug protocol; and increasing the dosage of 5-FU (or an analog or prodrug thereof) in the anticancer drug protocol. In this aspect, substituting 5,10-CH₂-THFA for leucovorin in the anticancer while increasing the dosage of 5-FU used in the regimen can increase the efficacy of a treatment without prohibitively increasing toxicity.

### EXAMPLES

### Example 1

### Nude Mouse Study on Colorectal Tumor HT-29 Treatment with 5-FU, 5,10-CH₂-THFA, Leucovorin, Anti-VEGF, and Oxaliplatin

Materials and Methods

### Mice

Nude (nu/nu) mice were obtained from Charles River Laboratories. Mice were 6-8 weeks old at the start of all studies. Mice were maintained in isolated, hepa-filter ventilated cages with 4 mice per cage at LAB International's vivarium (San Diego, Calif.).

### Cell Lines

The human colon carcinoma HT-29 was obtained from American Tissue Culture Collection (ATCC). Cell lines were maintained in DMEM containing 10% fetal bovine serum (FBS), 2 mM 1-glutamine, 100 units/ml penicillin, and 100 micrograms/ml streptomycin (DMEM-10) in a 37° C., 5% CO₂ humidified incubator. Cell lines were passaged every 2-3 days prior to in vivo experiments.

### Drugs

5-Fluorouracil (5-FU) was obtained from Calbiochem. Leucovorin (leucovorin) and oxaliplatin were obtained from Sigma-Aldrich. 5,10-CH₂-THFA was manufactured by Eprova A G. A monoclonal antibody to vascular endothelial growth factor (anti-VEGF) was either obtained from R&D Systems (clone 26503 recognizing the human VEGF isoform 165) or Genentech (Avastin).

HT-29 Colorectal Carcinoma Nude Mouse Study #1

HT-29 cells were prepared for injection as follows. Confluent tissue culture flasks of HT-29 cells were washed once with PBS followed by cell detachment with trypsin. Detached cells were then washed once in DMEM-10 followed by one wash with PBS. Finally, cells were resuspended at 2×10⁷ cells/ml in PBS. Nude mice (nu/nu) were inoculated subcutaneously with 100 microliters (2×10⁶ cells) of HT-29 cells using a 28 gauge insulin needle/syringe. When tumors reached 100 to 300 mm³ in volume, mice were treated with various combinations of 5-FU, 5,10-CH₂-THFA, leucovorin, oxaliplatin, and anti-VEGF (R&D Systems antibody) administered by intraperitoneal injection. All drugs were dosed daily (0.6 mg/mouse/drug) for five consecutive days with the exception of anti-VEGF and oxaliplatin. Anti-VEGF was dosed once (100 microgram/mouse) on day 5. Oxaliplatin was dosed once on day 1 (0.3mg/mouse). In addition, 5,10-CH₂-THFA or leucovorin were injected 20 minutes prior to 5-FU injection. Tumor sizes were measured every 2 to 3 days using calipers. Tumor volume was calculated using the following formula: tumor volume=(length×width²)/2. Mice were euthanized by CO₂ followed by cervical dislocation either when a tumor reached >2 cm in diameter or upon tumor ulceration.

### Data Analysis

Statistical analysis of tumor and blood data was performed using GraphPad Prism scientific software. Bonferonni's T test was used to compare tumor sizes between multiple groups. The Logrank test was used to determine statistical differences between group survival curves. In some cases, in which only two groups were compared, Student's T test was used to determine the significance between group measurements.

Results

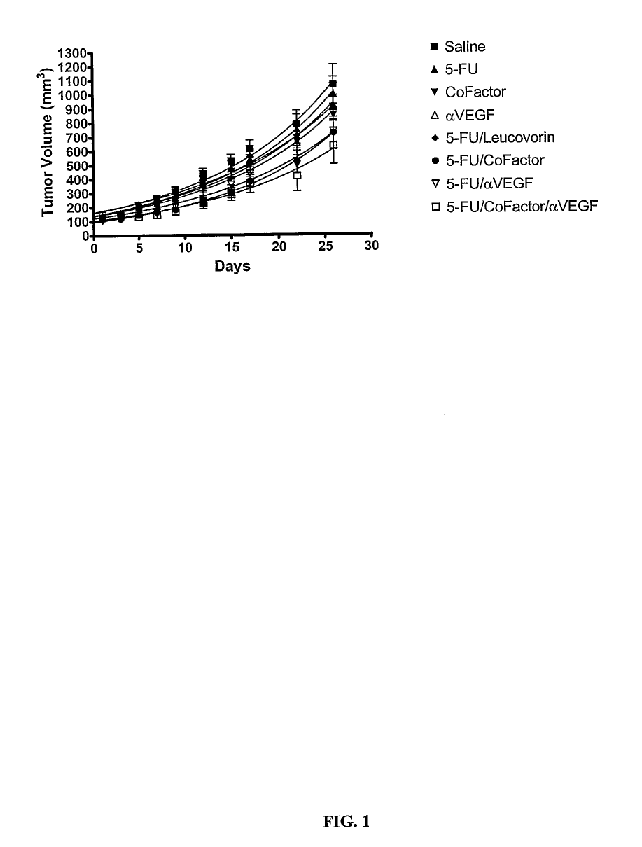

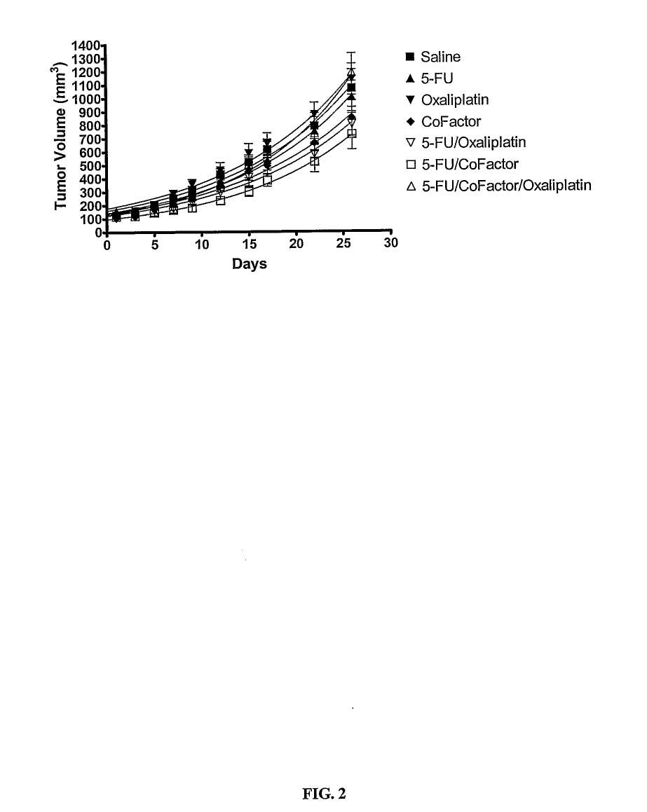

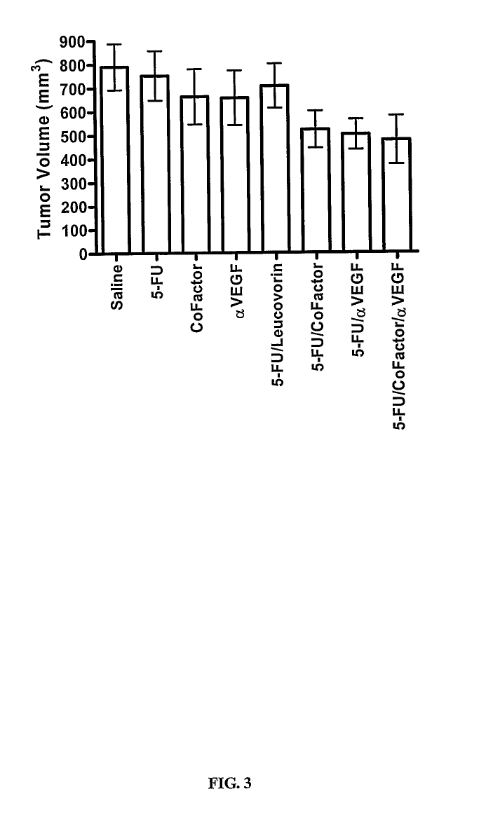

Nude mice were treated with the drug combinations described in Table 2. In this study, we wanted to examine if combining 5-FU/5,10-CH₂-THFA treatment with the oxaliplatin or anti-VEGF antibody (obtained from R&D Systems) could inhibit colorectal tumor growth more than other drug combinations. Drug concentrations and treatment days are described in the materials and methods section. Following treatment, tumor sizes were measured every 2-3 days and tumor volumes calculated. Tumor volumes were then plotted versus time from treatment initiation (FIGS. 1 and 2). To simplify the graphs, we divided analysis into graphs containing anti-VEGF data and another set with oxaliplatin data. Best-fit curves for each treatment group were calculated and plotted in these figures. As seen in FIG. 1, 5-FU/5,10-CH₂-THFA/anti-VEGF treated mice had the slowest tumor growth curve followed by either 5-FU/5,10-CH₂-THFA or 5-FU/anti-VEGF treated mice.

TABLE 2
Mouse Treatment Groups

| Group # | Treatment | Mice/group |
|---|---|---|
| 1 | Saline | 8 |
| 2 | 5-FU | 8 |
| 3 | 5,10-CH₂-THFA | 8 |
| 4 | Anti-VEGF | 8 |
| 5 | Oxaliplatin | 8 |
| 6 | 5-FU/Leucovorin | 8 |
| 7 | 5-FU/5,10-CH₂-THFA | 8 |
| 8 | 5-FU/anti-VEGF | 8 |
| 9 | 5-FU/Oxaliplatin | 8 |
| 10 | 5-FU/5,10-CH₂-THFA/anti-VEGF | 8 |
| 11 | 5-FU/5,10-CH₂-THFA/Oxaliplatin | 8 |
| Total | | 88 |

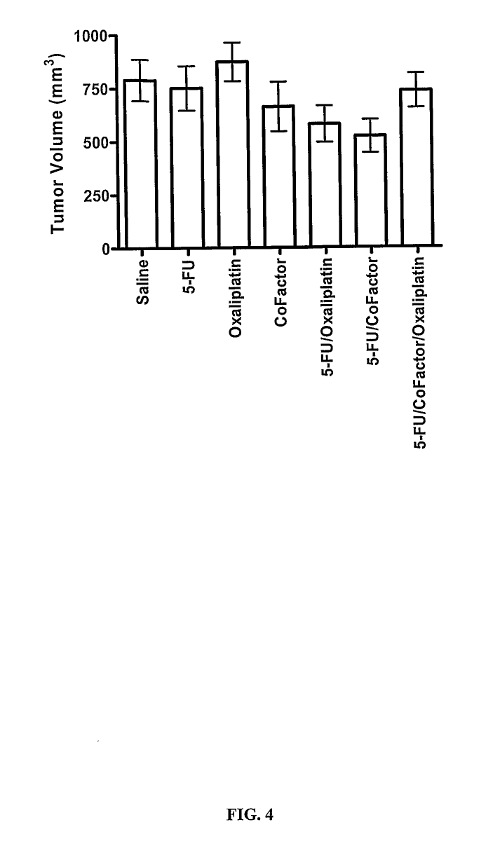

We also analyzed the differences between mean tumor volumes following treatment. Comparing the various treatment combinations for the anti-VEGF set of data (FIG. 3), we observed the mean tumor volume of 5-FU/5,10-CH₂-THFA/anti-VEGF treated mice (478.6±102.7, mean±SEM, n=7) was less than 5-FU (752.5±104.2, n=8), 5-FU/Leucovorin (707.5±93.6, n=8), 5-FU/5,10-CH₂-THFA (522.5±78.2, n=8), and 5-FU/anti-VEGF (502.5±64.1, n=8) treated mice. Oxaliplatin treated mice had the largest tumors (tumor volume 875.0±90.6, mean±SEM, n=8) (FIG. 4), indicating that the HT-29 tumor was not responsive to this drug (see Plasencia et al. (2002) American Society for Clinical Oncology Annual Meeting Abstract No. 2188.) The resistance of the HT-29 tumor to oxaliplatin probably accounts for the lack of equivalent tumor inhibition in the treatment group receiving the triple drug combination of 5-FU/5,10-CH₂-THFA /Oxaliplatin (735.0±80.3, n=8) (FIG. 4), when compared with the triple combination 5-FU/5,10-CH₂-THFA/anti-VEGF treated mice, which had the smallest tumor sizes of any anti-VEGF combination (FIG. 3).

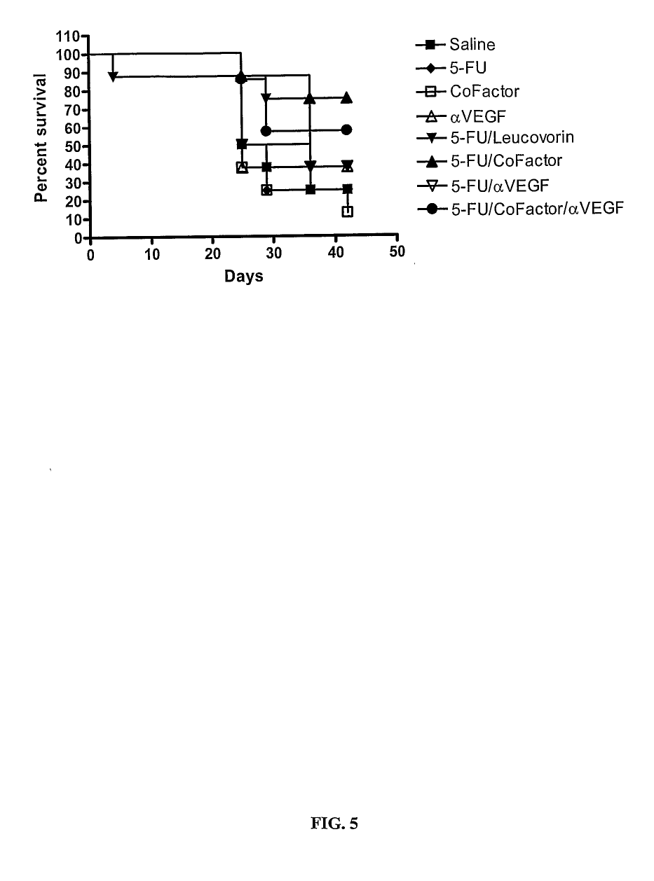

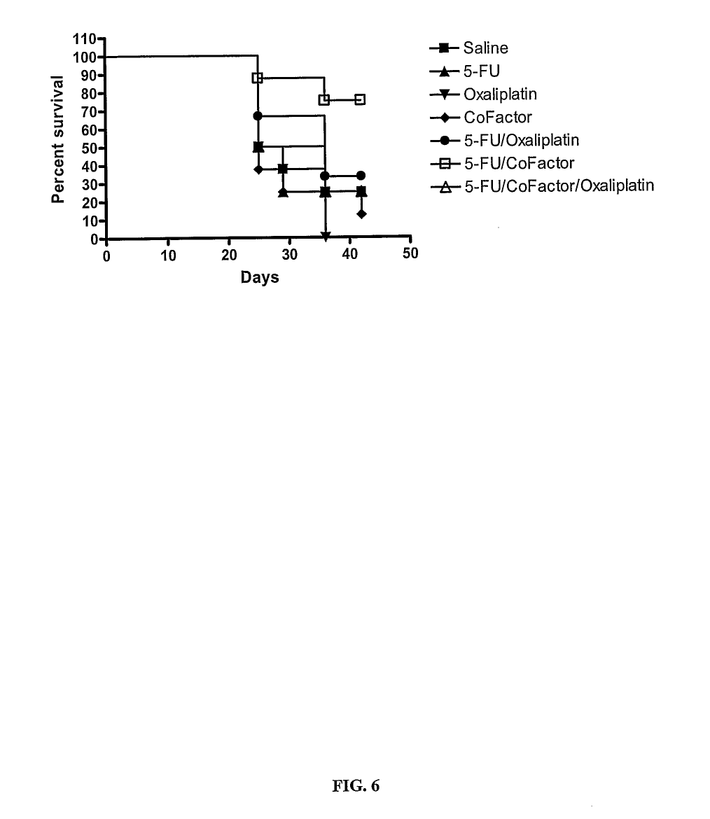

Mouse survival curves were also calculated for all treatment groups. Mice were euthanized upon overt systemic toxicity, tumor ulceration, or when tumor diameter reaches >2 cm. At the completion of the study period (42 days), 75% of mice treated with 5-FU/5,10-CH₂-THFA were still alive (FIG. 5). This survival was significantly longer than mice treated with only 5-FU (25%, p<0.05, Logrank test). In addition to 5-FU/5,10-CH₂-THFA treated mice, 5-FU/5,10-CH₂-THFA/anti-VEGF treated mice also survived longer (57%) than all other treatment groups. The lack of protection of mice treated with 5-FU/5,10-CH₂-THFA /Oxaliplatin (25%) (FIG. 6) compared to other treatment groups can most likely be attributed to the apparent resistance of the HT-29 tumor to oxaliplatin (FIG. 3). For the oxaliplatin treatment subgroup analysis, 5-FU/5,10-CH₂-THFA treatment provided the greatest survival benefit.

### Example 2

### Nude Mouse Study on Colorectal Tumor HT-29 Treatment with 5-FU, 5,10-CH₂-THFA, FA, and Anti-VEGF

Materials and Methods

### Mice

Nude (nu/nu) mice were obtained from Charles River Laboratories. Mice were 6-8 weeks old at the start of all studies. Mice were maintained in isolated, hepa-filter ventilated cages with 4 mice per cage at LAB International's vivarium (San Diego, Calif.).

### Cell Lines

The human colon carcinoma HT-29 was obtained from American Tissue Culture Collection (ATCC). Cell lines were maintained in DMEM containing 10% fetal bovine serum (FBS), 2 mM 1-glutamine, 100 units/ml penicillin, and 100 micrograms/ml streptomycin (DMEM-10) in a 37° C., 5% CO₂ humidified incubator. Cell lines were passaged every 2-3 days prior to in vivo experiments.

### Drugs

5-Fluorouracil (5-FU) was obtained from Calbiochem. Leucovorin (leucovorin) and oxaliplatin were obtained from Sigma-Aldrich. 5,10 methylenetetrahydofolate was manufactured by Eprova AG. A monoclonal antibody to vascular endothelial growth factor (anti-VEGF) was either obtained from R&D Systems (clone 26503 recognizing the human VEGF isoform 165) or Genentech (Avastin).

HT-29 Colorectal Carcinoma Nude Mouse Study #2

HT-29 cells were prepared for injection as follows. Confluent tissue culture flasks of HT-29 cells were washed once with PBS followed by cell detachment with trypsin. Detached cells were then washed once in DMEM-10 followed by one wash with PBS. Finally, cells were resuspended at 1×10⁷ cells/ml in PBS. Nude mice (nu/nu) were inoculated subcutaneously with 100 microliters (10⁶ cells) of HT-29 cells using a 28 gauge insulin needle/syringe. When tumors reached 30 to 100 mm³ in volume, mice were treated with various combinations of 5-FU, 5,10-CH₂-THFA, leucovorin, and anti-VEGF (Genentech's Avastin antibody) administered by intraperitoneal injection. All drugs were dosed daily (0.6 mg/mouse/drug) for seven consecutive days with the exception of anti-VEGF, dosed twice (100 micrograms/mouse) on days 1 and 7. In addition, 5,10-CH₂-THFA or leucovorin were injected 20 minutes prior to 5-FU injection. Tumor sizes were measured every 2 to 3 days using calipers. Tumor volume was calculated using the following formula: tumor volume=(length×width²)/2. Mice were euthanized by CO₂ followed by cervical dislocation either when a tumor reached >2 cm in diameter or upon tumor ulceration. cl Data Analysis

Statistical analysis of tumor and blood data was performed using GraphPad Prism scientific software. Bonferonni's T test was used to compare tumor sizes between multiple groups. The Logrank test was used to determine statistical differences between group survival curves. In some cases, in which only two groups were compared, Student's T test was used to determine the significance between group measurements.

Results

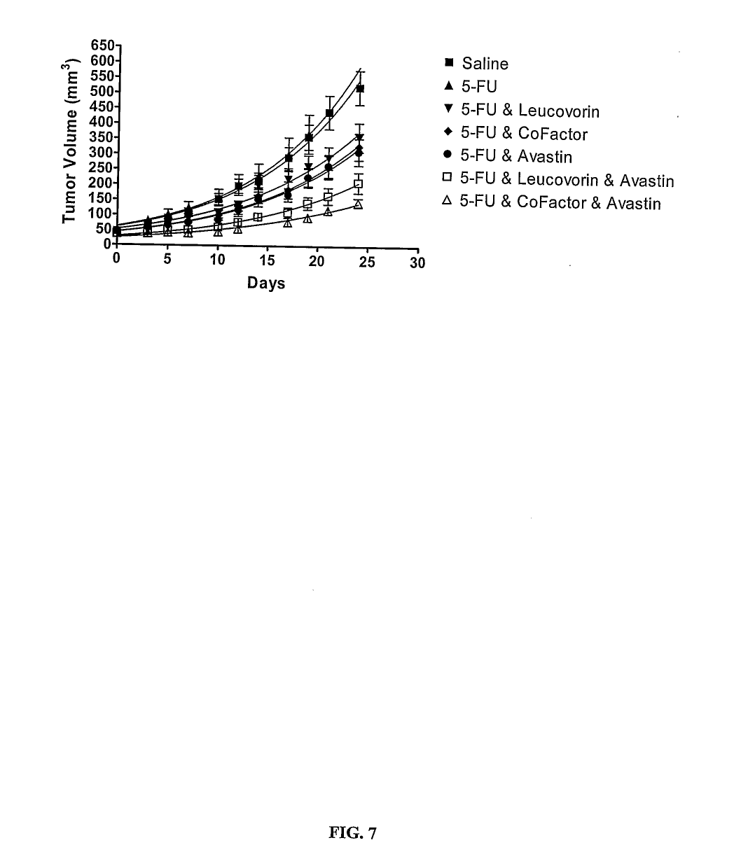

Based on the pilot results obtained in the first nude mouse study described above, we repeated another HT-29 nude mouse study with some modifications to study design. Modifications included larger group sizes, substitution of Genentech's anti-VEGF Avastin antibody for R&D System's antibody, exclusion of oxaliplatin, increased number of treatment days, and increased the number of doses of the anti-VEGF antibody. Nude mice were treated with the drug combinations described in Table 3. In this study, we wanted to examine if combining 5-FU/5,10-CH₂-THFA treatment with the anti-VEGF antibody Avastin could inhibit colorectal tumor growth more than other drug combinations. Drug concentrations and treatment days are described in the materials and methods section. Following treatment, tumor sizes were measured every 2-3 days and tumor volumes calculated. Tumor volumes were then plotted versus time from treatment initiation (FIG. 7). Best-fit curves for each treatment group were calculated and plotted in this figure. Based on the best-fit curve analysis, the average doubling time for each group was calculated (Table 4). Mice treated with the combination of 5-FU/5,10-CH₂-THFA/Avastin displayed the slowest growth kinetics (doubling time=9.9 days) compared to all other groups. These results are consistent with results obtained in the first nude mouse tumor study described earlier.

TABLE 3
Mouse Treatment Groups

| Group # | Treatment | Mice/group |
|---|---|---|
| 1 | Saline | 12 |
| 2 | 5-FU | 12 |
| 3 | 5-FU/Leucovorin | 12 |
| 4 | 5-FU/5,10-CH₂-THFA | 12 |
| 5 | 5-FU/Avastin | 12 |
| 6 | 5-FU/Leucovorin/Avastin | 12 |
| 7 | 5-FU/5,10-CH₂-THFA/Avastin | 12 |
| Total | | 84 |

TABLE 4
Tumor Doubling Times

| | | Doubling Time |
|---|---|---|
| Group # | Treatment | (days) |
| 1 | Saline | 7.6 |
| 2 | 5-FU | 7.4 |
| 3 | 5-FU/Leucovorin | 8.5 |
| 4 | 5-FU/5,10-CH₂-THFA | 8.2 |
| 5 | 5-FU/Avastin | 8.4 |
| 6 | 5-FU/Leucovorin/Avastin | 8.6 |
| 7 | 5-FU/5,10-CH₂-THFA/Avastin | 9.9 |

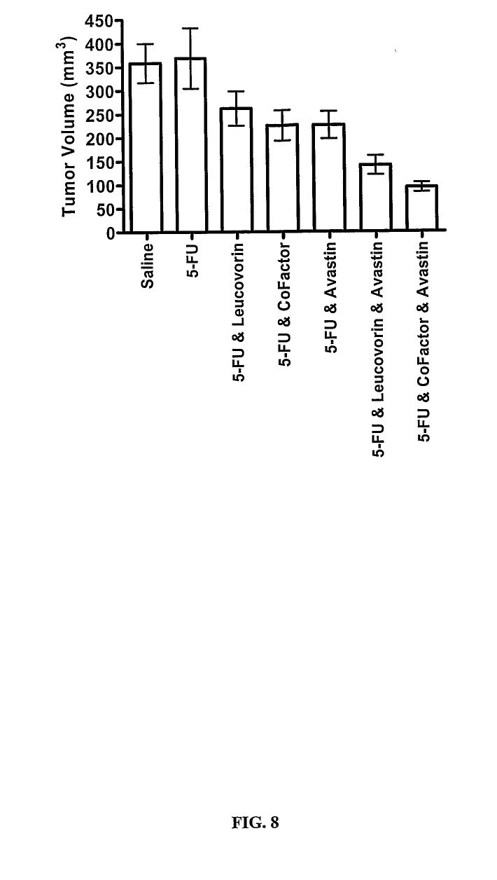

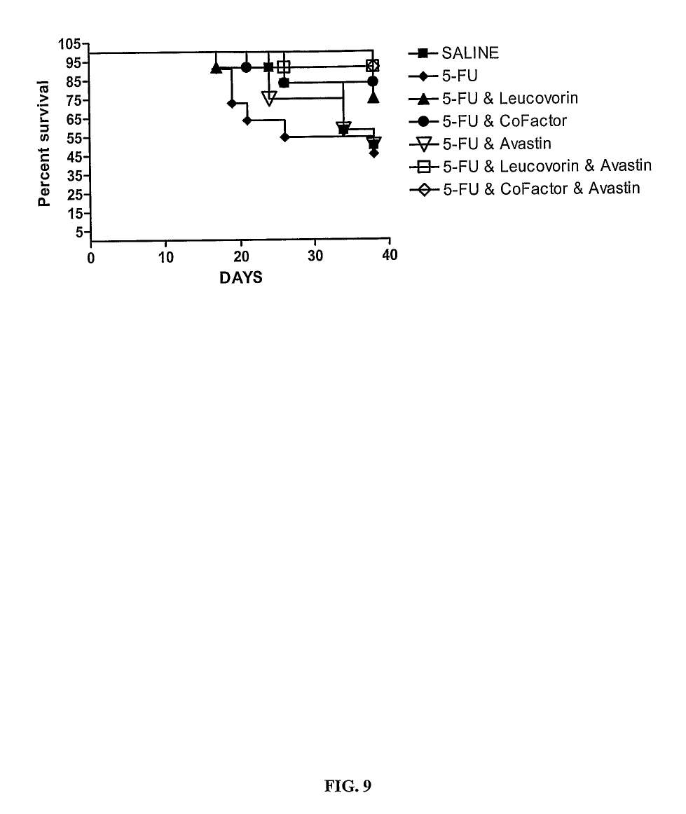

We also analyzed the differences between mean tumor volumes determined 19 days following treatment initiation. The mean tumor volumes±SEM are plotted in FIG. 8. We observed the mean tumor volume of 5-FU/5,10-CH₂-THFA/Avastin treated mice (94.0±10.2, mean±SEM, n=12) was significantly less (p<0.05, Bonferonni's T test) than 5-FU (368.5±63.7, n=10), 5-FU/Leucovorin (262.0±36.5, n=11), 5-FU/5,10-CH₂-THFA (225.4±32.0, n=12), 5-FU/Avastin (225.5±28.8, n=12), but not 5-FU/Leucovorin/Avastin (140.8±20.3, n=12) treated mice. In contrast, mean tumor volumes of 5-FU/Leucovorin/Avastin treated mice were only significantly smaller than tumor volumes in 5-FU treated mice but not other treatment groups.

Mouse survival curves were also calculated for all treatment groups. Mice were euthanized upon overt systemic toxicity, tumor ulceration, or when tumor diameter reached >2 cm. Prior to study completion (38 days from treatment initiation), ≦50% of mice treated with saline, 5-FU, or 5-FU plus Avastin were still alive (FIG. 9). In contrast, 92% of mice treated with 5-FU plus Avastin in combination with either 5,10-CH₂-THFA or leucovorin were still alive. This pattern of survival for the various drug combinations is similar to the results observed in the first nude mouse colorectal tumor study described above.

### Example 3

### Blood Analysis of Balb/c Mice Treated with Combinations of 5-FU, Leucovorin, and 5,10-CH₂-THFA

Materials and Methods

### Mice

Balb/c mice were obtained from Charles River Laboratories. Mice were 6-8 weeks old at the start of all studies. Mice were maintained in isolated, hepa-filter ventilated cages with 4 mice per cage at LAB International's vivarium (San Diego, Calif.).

### Drugs

5-Fluorouracil (5-FU) was obtained from Calbiochem. Leucovorin (folinic acid) was obtained from Sigma-Aldrich. 5, 10 methylenetetrahydofolate (5,10-CH₂-THFA) was manufactured by Eprova AG.

### Balb/c Blood Analysis Study

Balb/c mice, 7 weeks old female mice, were injected for seven consecutive days with combinations of 5-FU, leucovorin, and 5,10-CH₂-THFA. All drugs were intraperitoneally injected (100 microliters/mouse, 0.6mg/mouse/drug) using a 28 gauge insulin needle/syringe. 200-250 microliters blood/mouse was collected by retro-orbital puncture into EDTA-coated microtainer tubes (VWR International) on days 0 (prior to drug injection), 8, and 13. Complete blood counts plus blood differentials were determined by Labcorp Corporation of America using a Bayer Advia 120 Hematology analyzer.

Results

In addition to its tumoricidal activity, 5-FU is cytotoxic towards normal cells, especially cells of the hematopoietic system due to its myelosuppressive effects. Because of the related chemical characteristics and modes of action of leucovorin and 5,10-CH₂-THFA, we wanted to determine if there were similar toxicity profiles of 5-FU/5,10-CH₂-THFA combination therapy. As such, we injected normal Balb/c mice with various combinations of 5-FU, leucovorin, and 5,10-CH₂-THFA (Table 5). Pretreatment, one week, and two weeks following treatment, we analyzed complete blood counts plus differentials for changes in blood parameters. Furthermore, we analyzed qualitative and quantitative measures of drug toxicity.

TABLE 5
Balb/c Mouse Treatment Groups

| Group # | Treatment | Mice/group |
|---|---|---|
| 1 | 5-FU | 12 |
| 2 | 5-FU/Leucovorin | 13 |
| 3 | 5-FU/5,10-CH₂-THFA | 13 |
| Total | | 38 |

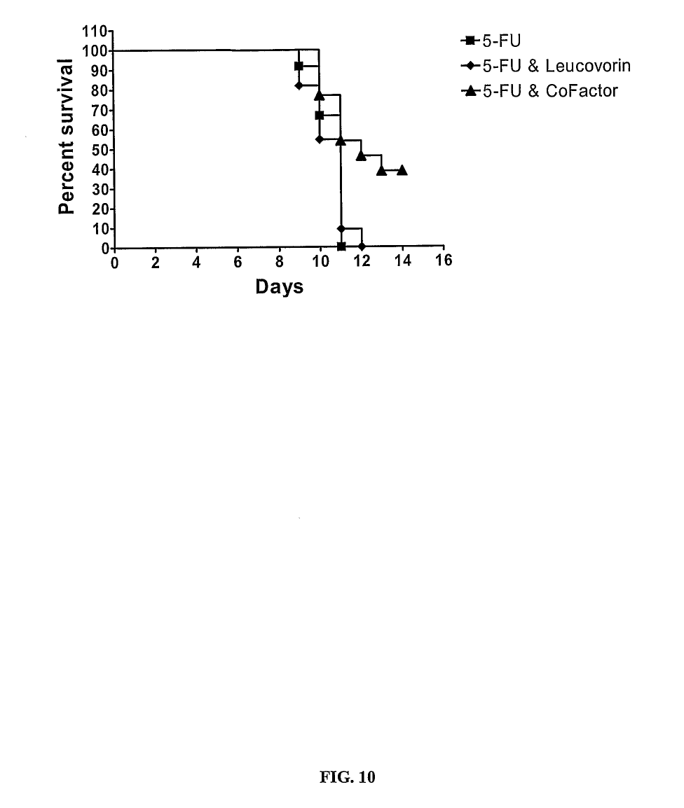

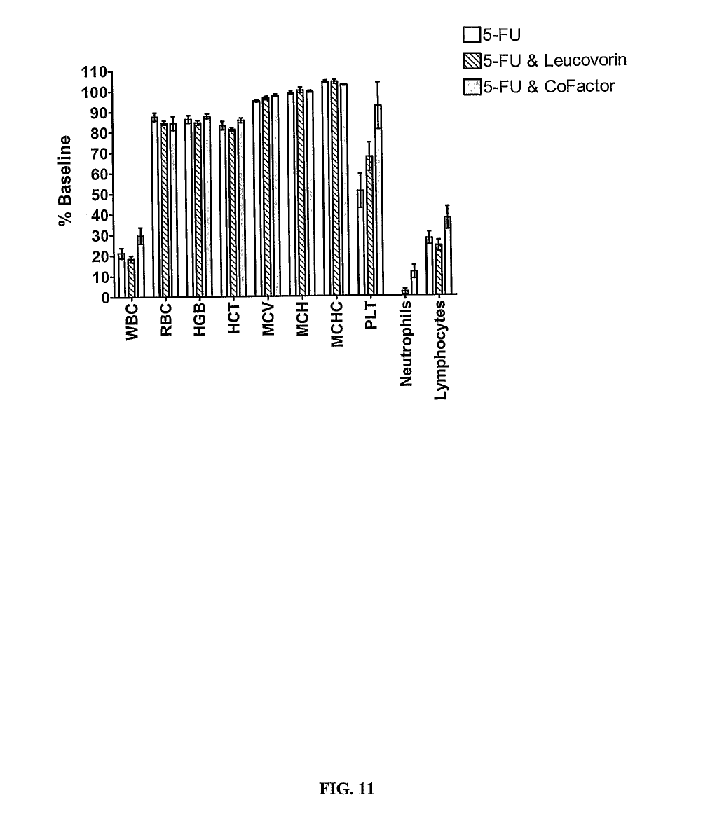

After one week of drug dosing, we observed all mice had drug-related toxicity including ruffled fur, moribundity, and dehydration. Within 12 days of initiation of drug treatment, all mice in the 5-FU only and 5-FU/leucovorin treatment groups had died. In contrast, 38% of mice (5 of 13) in the 5-FU/5,10-CH₂-THFA treatment group were alive after 14 days. Kaplan-Meier survival curves were plotted for all treatment groups (FIG. 10). Logrank statistical comparison of the 5-FU/5,10-CH₂-THFA treatment group versus the 5-FU/Leucovorin treatment group indicated a significant difference in survival (p<0.05).

Blood analysis also revealed differences in select blood cell types (FIG. 11). We measured the following blood parameters: white blood cells (WBC), red blood cells (RBC), hemoglobin (HGB), hematocrit (HCT), mean cell volume (MCV), mean cell hemoglobin (MCH), mean cell hemoglobin content (MCHC), neutrophils, lymphocytes, platelets (PLT), eosinophils, basophils, and monocytes. One week following drug treatment, we observed significantly more white blood cells in 5-FU/5,10-CH₂-THFA treated mice than 5-FU/leucovorin treated mice (p <0.05, Student's t test). Among the white blood cell subsets, we observed significantly more platelets and neutrophils in the 5-FU/5,10-CH₂-THFA treated group than the other treatment groups.

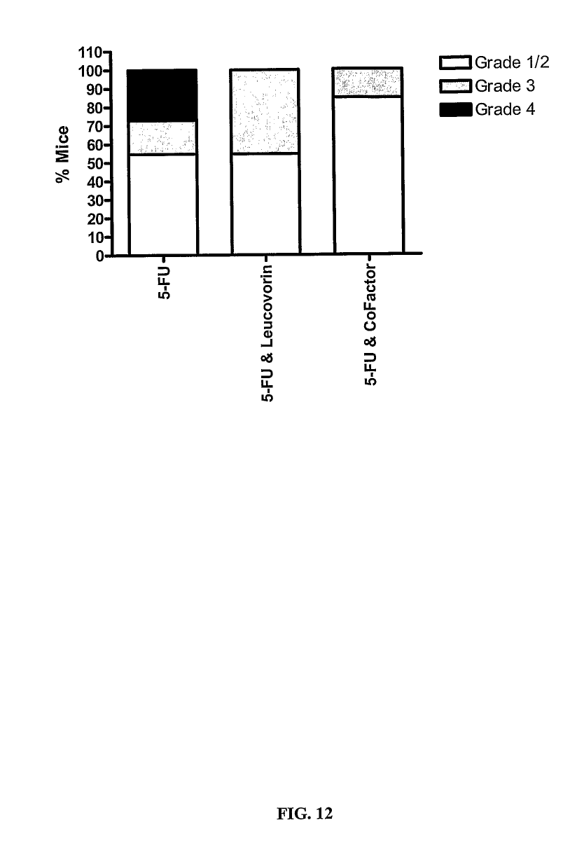

Since we observed differences in both platelet and neutrophil levels following 5-FU/5,10-CH₂-THFA treatment, we assessed these cell types further. Using NCI grading criteria for toxicity, we calculated the percentage of mice with either combined grade 1/2 toxicity, grade 3 toxicity, or grade 4 toxicity. For platelets, we observed 25% of mice treated 5-FU alone developed grade 4 toxicity (FIG. 12). In contrast, no grade 4 toxicity was noted for either 5-FU/leucovorin or 5-FU/5,10-CH₂-THFA treated mice. However, unlike 5-FU/leucovorin mice with grade 3 toxicity (45%), only 15% of 5-FU/5,10-CH₂-THFA treated mice developed grade 3 platelet toxicity. The remaining 5-FU/5,10-CH₂-THFA treated mice (85%) developing only grade 1 or 2 toxicity. As such, this data suggests 5-FU/5,10-CH₂-THFA induces milder platelet toxicity than either 5-FU alone or 5-FU/leucovorin.

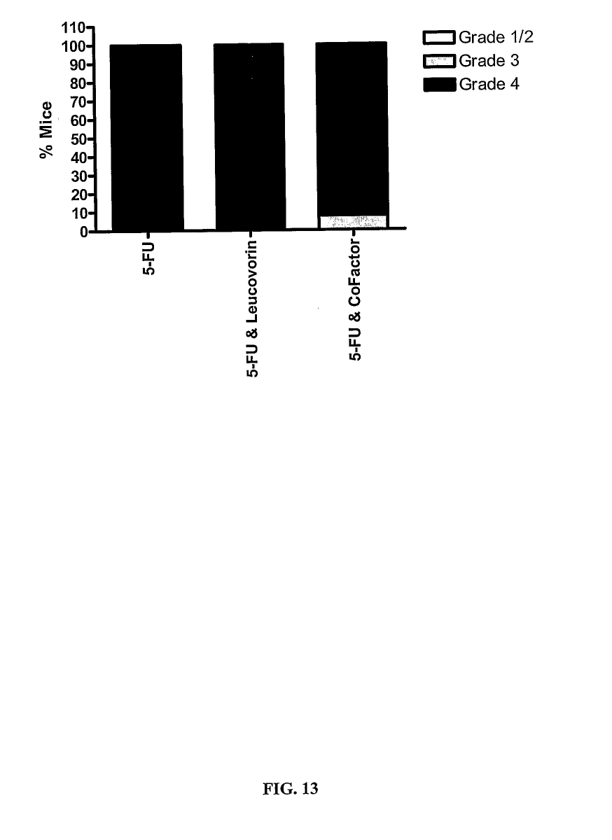

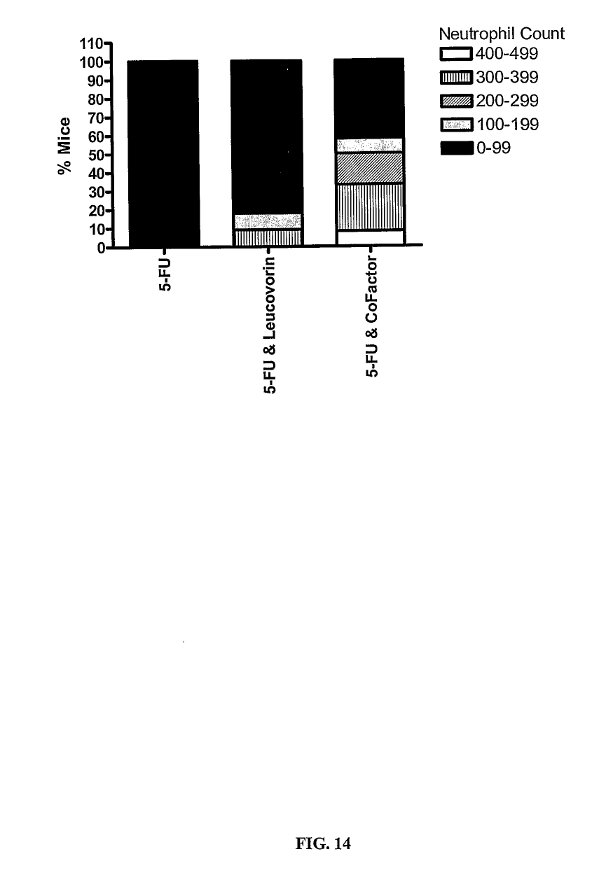

Similarly, we assessed the neutrophil toxicity profiles. In contrast to the platelet differences, the standard NCI grading system did not reveal noticeable neutrophil differences between treatment groups. For example, 100% of both 5-FU only and 5-FU/leucovorin treated mice developed grade 4 toxicity while 92% of 5-FU/5,10-CH₂-THFA treated mice developed the same grade 4 toxicity. The remaining 8% of 5-FU/5,10-CH₂-THFA treated mice developed grade 3 toxicity (FIG. 13). However, closer analysis of mice that developed grade 4 toxicity revealed quantifiable neutrophil differences. We divided mice with grade 4 toxicity into subgroups based on their neutrophil cell count ranges following treatment (FIG. 14). This analysis revealed that 100% of mice treated with 5-FU only, and 80% of 5-FU/leucovorin treated mice, had neutrophil cell counts between 0 and 99. In contrast, only 40% of 5-FU/5,10-CH₂-THFA treated mice developed this lowest level neutrophil cell count. The majority of grade 4-rated 5-FU/5,10-CH₂-THFA treated mice (50%) had neutrophil cell counts in the range of 200-499. Thus, this data suggests 5-FU/5,10-CH₂-THFA results in milder neutrophil toxicity than either 5-FU alone or 5-FU/leucovorin.

### Example 4

### Weight Loss Toxicity Analysis of Balb/c Mice Treated with Combinations of 5-FU, Leucovorin, 5,10-CH₂-THFA, and Gemcitabine

Materials and Methods

### Mice

Balb/c mice were obtained from Charles River Laboratories. Mice were 6-8 weeks old at the start of the study. Mice were maintained in isolated, hepa-filter ventilated cages with 4 mice per cage at LAB International's vivarium (San Diego, Calif.).

### Drugs

5-Fluorouracil (5-FU) and leucovorin (leucovorin) were obtained from Sigma-Aldrich. 5,10 methylenetetrahydofolate (5,10-CH₂-THFA) was manufactured by Eprova AG. Gemcitabine was manufactured by Eli Lilly and purchased from Myoderm Inc.

### Balb/c Weight Analysis Study

Balb/c female mice were injected with combinations of 5-FU, leucovorin, 5,10-CH₂-THFA, and gemcitabine. 5-FU, leucovorin, and 5,10-CH₂-THFA were intraperitoneally injected (100 microliters/mouse, 0.6 mg/mouse/drug) for five consecutive days (days 1-5). Gemcitabine was intraperitoneally injected (100 microliters/mouse, 100 micrograms/mouse) every three days (days 1, 4, and 7). All drugs were injected using a 27 gauge insulin needle/syringe. Mouse weights were measured using an analytical balance prior to initiation of drug dosing (pretreatment) and on day 8.

Results

A known toxicity of 5-FU is gastrointestinal toxicity and associated weight loss. It is reported that leucovorin can potentially exacerbate gastrointestinal toxicity. Furthermore, gemcitabine, the current standard therapy for pancreatic cancer, has its own associated toxicity profile. While combination 5-FU/gemcitabine and 5-FU/leucovorin/gemcitabine therapy have been examined in the clinic and shown to have enhanced clinical activity, these combinations typically display more severe toxicity than gemcitabine alone or 5-FU/leucovorin alone. Because of the related chemical characteristics and modes of action of leucovorin and 5,10-CH₂-THFA, we wanted to investigate the toxicity profiles of 5-FU/5,10-CH₂-THFA in combination with gemcitabine, since 5-FU/5,10-CH₂-THFA/gemcitabine combination therapy is a potential treatment regimen for pancreatic cancer. Furthermore, we wanted to expand upon our previous toxicity analysis of combination 5-FU/5,10-CH₂-THFA and determine if this combo has additional non-obvious toxicity profiles compared to either 5-FU/leucovorin or 5-FU alone. As such, we injected normal Balb/c mice with various combinations of 5-FU, leucovorin, 5,10-CH₂-THFA, and gemcitabine (Table 6). Pretreatment and one week following treatment initiation, we examined weight loss/gain as a measure of gastrointestinal toxicity.

TABLE 6
Balb/c Mouse Treatment Groups

| Group # | Treatment | Mice/group |
|---|---|---|
| 1 | 5-FU | 11 |
| 2 | 5-FU/Leucovorin | 12 |
| 3 | 5-FU/5,10-CH₂-THFA | 12 |
| 4 | Gemcitabine | 12 |
| 5 | 5-FU/Leucovorin/Gemcitabine | 12 |
| 6 | 5-FU/5,10-CH₂-THFA/Gemcitabine | 12 |
| Total | | 71 |

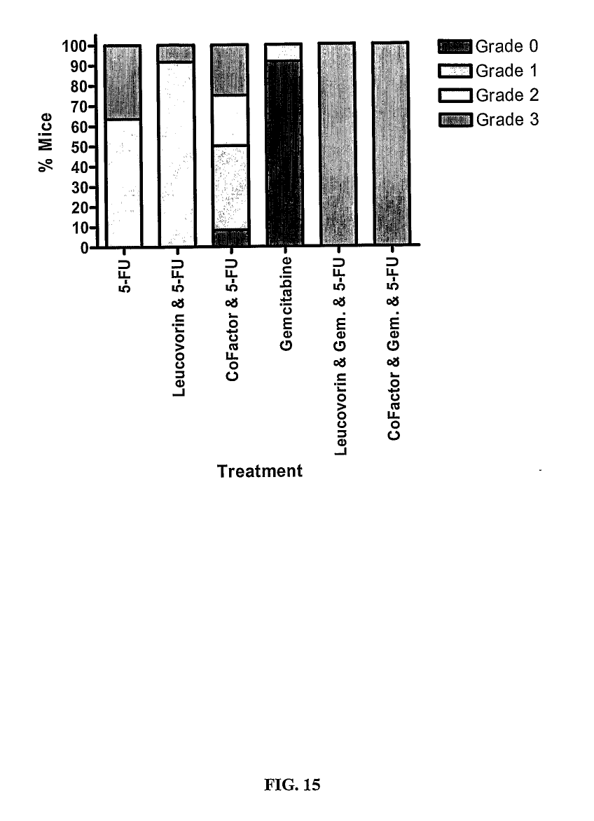

Prior to initiation of drug administration (pre-treatment), randomized groups of mice (12 per group) displayed similar mean body weights. Following treatment (day 8), mouse weights decreased in all treatment groups. Using the National Cancer Institute's (NCI) Common Terminology Criteria for Adverse Events, the severity of weight loss was plotted for each treatment group (FIG. 15). Toxicity grading is based on the percentage weight loss from the starting baseline weight (Table 7). These results show 5-FU/5,10-CH₂-THFA induced significantly less (p <0.05, Fisher's exact test) grade 2-3 toxicity (50%) than either 5-FU alone or combination 5-FU/leucovorin treatment (100% grade 2-3 toxicity for both treatment groups).

TABLE 7
National Cancer Institute Weight Loss Toxicity Grades

Toxicity

| | Grade 0 | Grade 1 | Grade 2 | Grade 3 |
|---|---|---|---|---|
| Weight Loss | <5% | 5-<10% | 10-<20% | ≧20% |

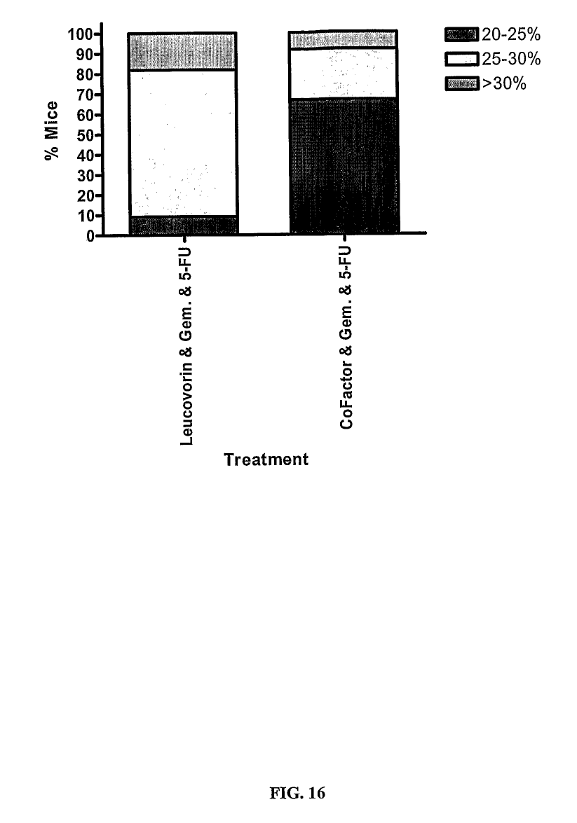

While gemcitabine treatment alone did not induce weight loss toxicity greater than grade 1 due to administration of a subtoxic concentration, addition of gemcitabine to either 5-FU/leucovorin or 5-FU/5,10-CH₂-THFA treatment resulted in 100% of mice with grade-3 toxicity (FIG. 15). However, quantitative differences in the percentage weight loss could be detected between these treatment groups (FIG. 16). This data suggests 5,10-CH₂-THFA protects mice from weight loss more effectively than leucovorin when used in combination with dual-cytotoxic drugs 5-FU and gemcitabine. While 92% of 5-FU/leucovorin/gemcitabine treated mice had >25% weight loss, significantly less (p<0.05, Fisher's exact test) 5-FU/5,10-CH₂-THFA/gemcitabine treated mice had this severity of weight loss (33% of mice).

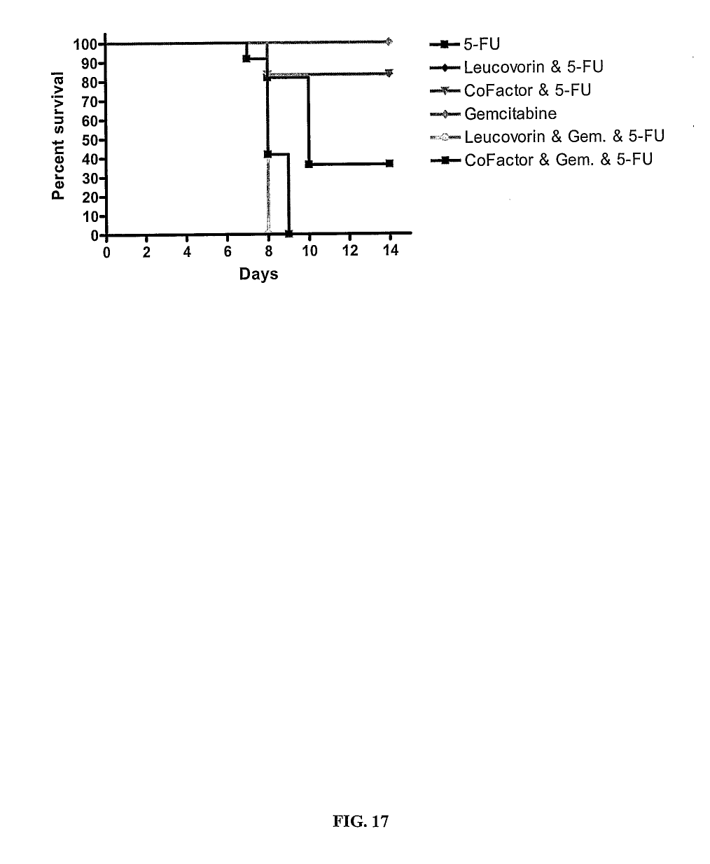

Mouse survival was also followed over time for each treatment group (FIG. 17). 5-FU/leucovorin and 5-FU/5,10-CH₂-THFA groups both had significantly greater percentages (p<0.05, Logrank test) of mice survive for up to 14 days (83% for each group), compared to mice treated with only 5-FU only (36%). The shortest survival time was observed in the triple drug combinations of either 5-FU/leucovorin/gemcitabine or 5-FU/5,10-CH₂-THFA /gemcitabine in which 100% of the mice died prior to day 14. However, 5-FU/5,10-CH₂-THFA/gemcitabine mice did survive significantly longer (9 days, p<0.05, Logrank test) than 5-FU/leucovorin/gemcitabine treated mice (8 days). This correlates with the less severe weight loss toxicity described above for the 5-FU/5,10-CH₂-THFA/gemcitabine combination group, and again suggests 5,10-CH₂-THFA induces milder weight loss compared to leucovorin when used with combination 5-FU/gemcitabine regimens.

### Example 5

### Lymphocyte Analysis of Balb/c Mice Treated with Combinations of 5-FU, Leucovorin, and 5,10-CH₂-THFA

Materials and Methods

### Mice

Balb/c mice were obtained from Charles River Laboratories. Mice were 6-8 weeks old at the start of all studies. Mice were maintained in isolated, hepa-filter ventilated cages with 4 mice per cage at LAB International's vivarium (San Diego, Calif.).

### Drugs

5-Fluorouracil (5-FU) was obtained from Calbiochem. Leucovorin (leucovorin) was obtained from Sigma-Aldrich. 5,10 methylenetetrahydofolate (5,10-CH₂-THFA) was manufactured by Eprova AG.

Balb/c Blood Analysis Study

Balb/c mice, 7 weeks old female mice, were injected for seven consecutive days with combinations of 5-FU, leucovorin, and 5,10-CH₂-THFA. All drugs were intraperitoneally injected (100 microliters/mouse, 0.6mg/mouse/drug) using a 28 gauge insulin needle/syringe. 200-250 microliters blood/mouse was collected by retro-orbital puncture into EDTA-coated microtainer tubes (VWR International) on days 0 (prior to drug injection), 8, and 13. Complete blood counts plus blood differentials were determined by Labcorp Corporation of America using a Bayer Advia 120 Hematology analyzer.

Results

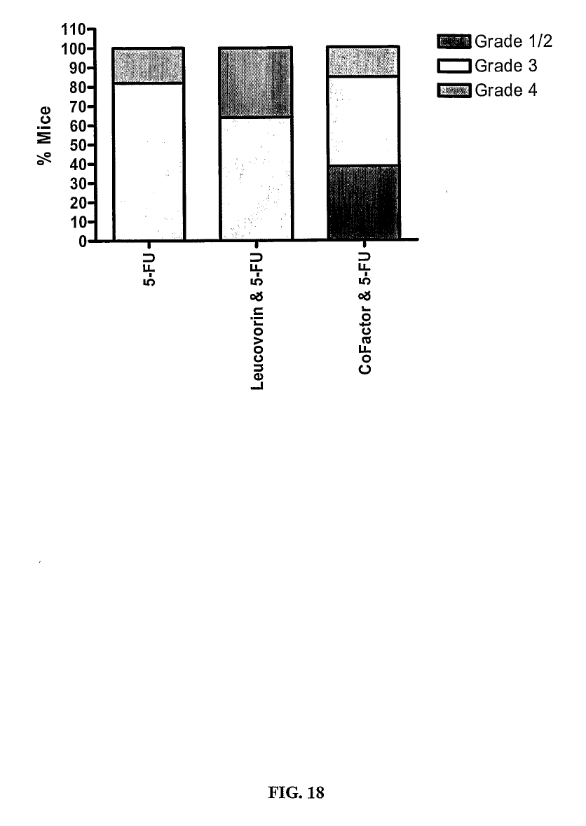

Additional analysis of the experiment described in Example 3 has revealed further toxicity differences between treatments groups. As originally described, we noted protection in white blood cells, including platelets and neutrophils, in the 5 -FU/5,10-CH₂-THFA treatment group compared to 5-FU/leucovorin and 5-FU alone. New analysis of the data, using NCI toxicity grading based on the percentage of baseline lymphocyte levels (Table 8), also shows greater protection of lymphocytes in the 5-FU/5,10-CH₂-THFA treatment group compared to the other groups (FIG. 18). While 100% of mice in the 5-FU only and 5-FU/leucovorin treatment groups developed Grade 3-4 lymphopenia, significantly less (p<0.05, Fisher's exact test) mice in the 5-FU/5,10-CH₂-THFA treatment group developed this level of toxicity (62%). As such, this data suggests 5-FU/5,10-CH₂-THFA induces milder lymphocyte toxicity than either 5-FU alone or 5-FU/leucovorin.

TABLE 8
National Cancer Institute Lymphopenia Toxicity Grades

Toxicity

| | Grade 1 | Grade 2 | Grade 3 | Grade 4 |
|---|---|---|---|---|
| Lympho- | 75-<100% | 50-<75% | 25-<50% | <25% |
| penia | LLN | LLN | LLN | LLN |

### Example 6

### Nude Mouse Study on HT-29 Colorectal Tumor Treatment with Capecitabine (Xeloda), 5,10-methylenetetrahydrofolate (5,10-CH₂-THFA), and Leucovorin

Materials and Methods

### Mice

Nude (nu/nu) mice were obtained from Simonsen Laboratories. Mice were 6-8 weeks old at the start of all studies. Mice were maintained in isolated, hepa-filter ventilated cages with 4 mice per cage at Perry Scientific's vivarium (San Diego, Calif.).

### Cell Lines

The human colon carcinoma HT-29 was obtained from American Tissue Culture Collection (ATCC). Cells were maintained in DMEM containing 10% fetal bovine serum (FBS), 2 mM 1-glutamine, 100 units/ml penicillin, and 100 micrograms/ml streptomycin (DMEM-10) in a 37° C., 10% CO₂ humidified incubator. Cells were passaged every 2-3 days prior to in vivo experiments.

### Drugs

Capecitabine (Xeloda) was manufactured by Roche Laboratories (Nutley, N.J.) Leucovorin (leucovorin) was obtained from Sigma-Aldrich. 5,10 methylenetetrahydofolate (5,10-CH₂-THFA) was manufactured by Eprova AG.

### Treatment

HT-29 cells were prepared for injection as follows: Confluent tissue culture flasks of HT-29 cells were washed once with PBS followed by cell detachment with trypsin. Detached cells were then washed once in DMEM-10 followed by one wash with PBS. Finally, cells were resuspended in PBS at 10⁷ cells/ml. Nude mice (nu/nu) were inoculated subcutaneously with 100 microliters (10⁶ cells) of HT-29 cells using a 28 gauge needle/1 ml insulin syringe. When tumors reached 100 to 300 mm³ in volume, mice were treated with various combinations of Xeloda, 5,10-CH₂-THFA, leucovorin, or water. Water and Xeloda (72mg/mouse/day) were administered by oral gavage. 5,10-CH₂-THFA and leucovorin were administered by intraperitoneal injection (0.6 mg/mouse/drug/day) approximately 20 minutes prior to Xeloda. All drugs were dosed daily for fourteen consecutive days. Tumor sizes and mouse body weights were measured every 2-4 days. Tumor volume was calculated using the following formula: tumor volume=(length×width²)/2. Mice were euthanized by CO₂ followed by cervical dislocation either when a tumor reached >2cm in diameter or upon tumor ulceration.

### Data Analysis

Statistical analysis and curve fitting of tumor growth, survival, and weight loss was performed using GraphPad Prism scientific software.

Results

### Tumor Growth

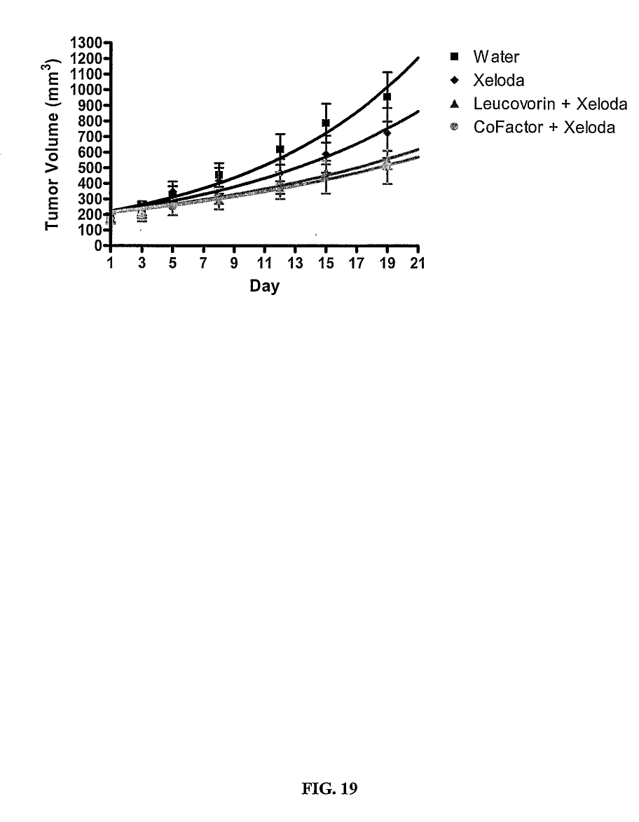

Tumor-bearing mice were treated with combinations of drugs shown in Table 9. Xeloda was dosed orally similar to the clinical regimen approved for human use. Compared to control treated mice (Water), all Xeloda-containing treatment groups had slower tumor growth (FIG. 19). Furthermore, both leucovorin and 5,10-CH₂-THFA increased anti-tumor activity of Xeloda. These differences can be seen in the tumor doubling times, calculated from the best-fit linear regression of exponential tumor growth, shown in Table 10.

TABLE 9
Mouse Treatment Groups

| Group | Treatment |
|---|---|
| 1 | Water |
| 2 | Xeloda |
| 3 | Leucovorin + Xeloda |
| 4 | 5,10-CH₂-THFA + Xeloda |

TABLE 10
Tumor Doubling Times

| Group | Treatment | Doubling Time (Days) |
|---|---|---|
| 1 | Water | 8.2 |
| 2 | Xeloda | 10.1 |
| 3 | Leucovorin + Xeloda | 13.2 |
| 4 | 5,10-CH₂-THFA + Xeloda | 14.2 |

### Survival

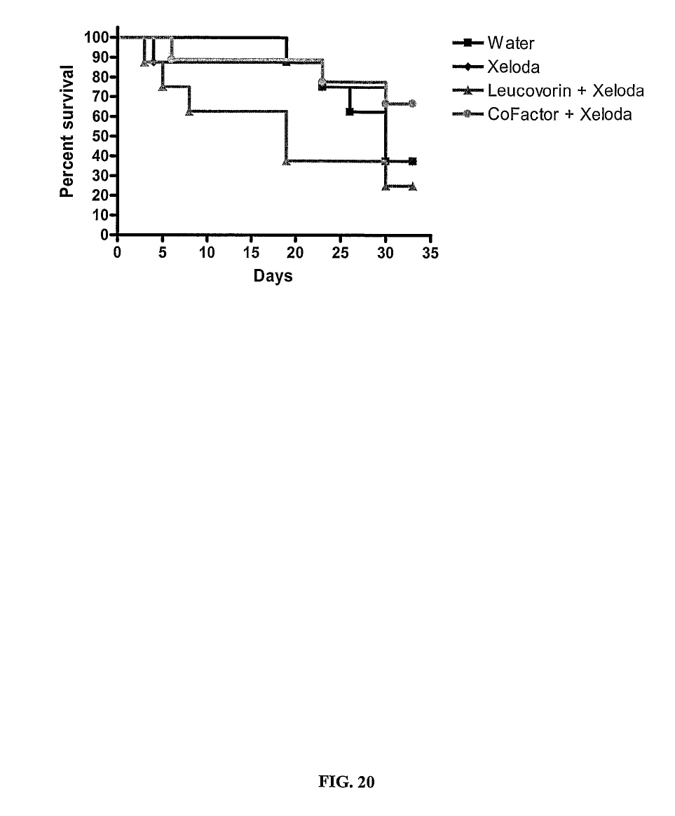

Mouse survival was followed throughout the course of the experiment (FIG. 20). These results indicated 5,10-CH₂-THFA plus Xeloda resulted in the greatest survival (67%) on day 33 of the experiment, with day 1 defined as the first day of drug dosing, compared to leucovorin plus Xeloda (25%) or Xeloda alone (38%). Furthermore, this data suggests mice treated with leucovorin plus Xeloda had a more rapid mortality rate as indicated by a median survival of 19 days compared to >30 days for all other treatment groups.

### Drug Toxicity

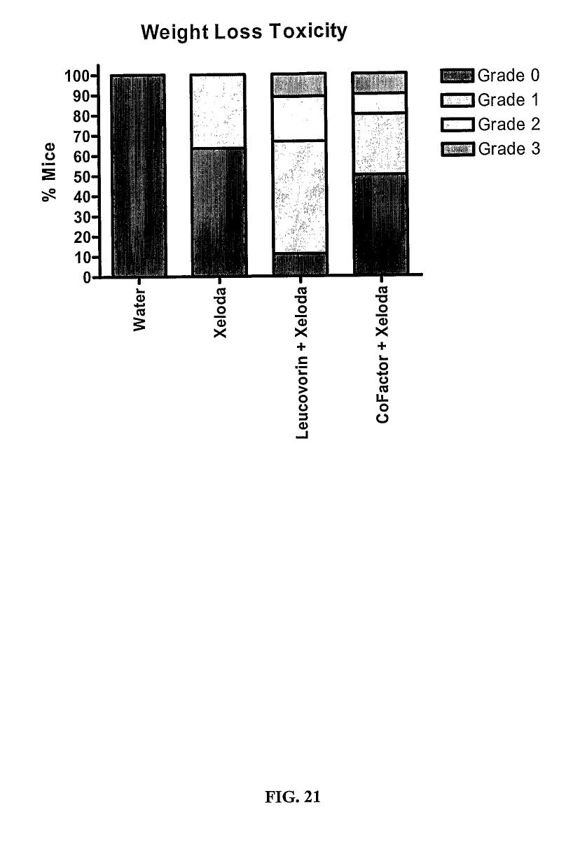

As a surrogate marker for drug toxicity, we examined mouse body weights over time. Using the National Cancer Institute's Common Toxicity Criteria version 3 grading system for weight loss (Table 11), the maximum toxicity grade of weight loss was plotted (FIG. 21). While Xeloda by itself was relatively nontoxic, inducing only grade 1 toxicity in 36% of the mice, leucovorin increased the overall grade 1-3 toxicity to 90% of mice. This increased toxicity is consistent with phase II human clinical trial results showing leucovorin increased Xeloda toxicity parameters such as diarrhea, vomiting, and mucosal inflammation (Van Cutsem, E., M. Findlay, B. Osterwalder, W. Kocha, D. Dailey, R. Pazdur, J. Cassidy, L. Dirix, C. Twelves, D. Allman, J. F. Seitz, J. Scholmerich, H. U. Burger, and J. Verweij. 2000. Capecitabine, an oral fluoropyrimidine carbamate with substantial activity in advanced colorectal cancer: results of a randomized phase II study. J Clin Oncol 18:1337). In contrast, 5,10-CH₂-THFA did not increase Xeloda toxicity in the mice as much as leucovorin, with only 50% of mice with grade 1-3 weight loss, a 40% reduction in toxicity compared to the leucovorin treatment group.

TABLE 11
Weight Loss Toxicity Criteria

Grade

| | 0 | 1 | 2 | 3 |
|---|---|---|---|---|
| % Weight Loss | <5% | 5-<10% | 10-<20% | ≧20% |

### CONCLUSIONS

Together, this data suggests 5,10-CH₂-THFA enhances Xeloda anti-tumor efficacy with less toxicity than leucovorin.

### Bibliography

U.S. Pat. No. 5,376,658 issued Dec. 27, 1994 to Spears et al.
U.S. Pat. No. 5,534,519 issued Jul. 9, 1996 to Spears et al.
Carlsson et al. (1997) The Cancer Journal 10: 266-273.
Plasencia, Taron, Martinez, McLeod, Rosell, and Abad (2002) Molecular aspects involved in chemotherapy response in sensitive and 5FU resistant colorectal cancer (CRC) cell lines. American Society for Clinical Oncology Annual Meeting Abstract No. 2188.

All headings are for the convenience of the reader and should not be used to limit the meaning of the text that follows the heading, unless so specified.

All references cited herein, including those in the bibliography, are incorporated by reference in their entireties.

1-257. (canceled)

258. A method of treating a patient with a cancerous tumor, the method comprising co-administering to the patient: (i) a thymidilate synthase (TS) inhibitor in combination with 5,10 methylene tetrahydrofolate; and, (ii) an anti-VEGF antibody, wherein the TS inhibitor and the anti-VEGF antibody are administered in dosage amounts effective to reduce the volume of the tumor.

259. The method of claim 258, wherein the TS inhibitor is 5-fluorouracil (5-FU) or an analogue or prodrug of 5-FU.

260. The method of claim 259, wherein the TS inhibitor is administered intravenously, or by injection, or orally.

261. The method of claim 259, wherein the TS inhibitor is 5-FU and the dosage amount of the 5-FU is from about 100 milligrams to about 1 gram per m².

262. The method of claim 259, wherein the prodrug is N4-pentyloxylcarbonyl-5′-deoxy-5-fluorocytidine (capecitabine).

263. The method of claim 262, wherein the dosage amount of the capecitabine is from about 1000 mg to about 5 grams per m².

264. The method of claim 258, wherein the 5,10 methylene tetrahydrofolate is administered in-travenously or by injection.

265. The method of claim 258, wherein the dosage amount of the 5,10 methylene tetrahydrofolate is from about 50 milligrams to about 250 milligrams per m².

266. The method of claim 258, wherein the tumor is colorectal cancer, breast cancer, gastric cancer, non-small-cell lung cancer, cervical cancer, ovarian cancer, pancreatic cancer, esophageal cancer, or head-and-neck cancer.

267. The method of claim 258, wherein the anti-VEGF antibody is bevacizumab (Avastin). administration.

268. A method of treating a patient with a cancerous tumor, the method comprising co-administering to the patient the following combination of drugs:
(i) N4-pentyloxylcarbonyl-5′-deoxy-5-fluorocytidine (capecitabine);
(ii) 5,10 methylene tetrahydrofolate; and
(iii) at least one additional chemotherapeutic agent selected from the group consisting of:
an alkylating agent, an antimetabolite, a topoisomerase inhibitor, a microtubule disrupting drug, a nucleic acid synthesis inhibitor, a kinase inhibitor, a hormone blocking drug, a proteosome inhibitor, a vascularization inhibitor, an immune modulator, an anti-inflammatory, a cytokine, an inhibitor of a cytokine, a receptor-binding drug, and a 5-fluorouracil modulator; wherein the combination of drugs are administered in dosage amounts effective to reduce the volume of the tumor.

269. The method of claim 268 wherein the cancer being treated is colorectal cancer, breast cancer, gastric cancer, non-small-cell lung cancer, cervical cancer, ovarian cancer, pancreatic cancer, esophageal cancer, or head-and-neck cancer.

270. The method of claim 268, wherein the at least one additional chemotherapeutic agent is a specific binding member, or a nucleic acid or a nucleic acid analogue molecule, or a small molecule.

271. The method of claim 270, wherein said specific binding member comprises an antibody that binds a growth factor.

272. The method of claim 271, wherein said antibody that binds a growth factor is at least one antibody that binds VEGF.

273. The method of claim 272, wherein the antibody the binds VEGF is bevacizumab.

274. The method of claim 271, wherein the antibody that binds a growth factor is at least one antibody that binds EGFR.

275. The method of claim 274, wherein the antibody that binds EGFR is cetuximab.

276. The method of claim 268, wherein the at least one additional chemotherapeutic agent is selected from the group comprising: irinotecan (CPT-11), difluorodeoxycytidine (gemcitabine), (E)-2′-deoxy-2′-(fluoromethylene) cytidine (tezacitabine), doxorubicin, epirubicin, mitomycin C, cyclophosphamide, cisplatin, oxaliplatin, paclitaxel, docetaxel, vincristine, vinblastine and vinorelbine.

277. The method of claim 268, wherein the combination of drugs are are formulated separately.

278. The method of claim 268, wherein combination of drugs is formulated for oral administration.
